(19) United States
(12) Patent Application Publication
(10) Pub. No.: US 2018/0051336 A1
(43) Pub. Date: Feb. 22, 2018

(54) METHODS FOR DIAGNOSING MULTIPLE SCLEROSIS USING VH4 ANTIBODY GENES

(71) Applicant: AMARANTUS BIOSCIENCE HOLDINGS, INC., San Francisco, CA (US)

(72) Inventor: Tom WILKS, Silver Spring, MD (US)

(21) Appl. No.: 15/546,171

(22) PCT Filed: Feb. 5, 2016
(86) PCT No.: PCT/US16/16862

§ 371 Date: Jul. 25, 2017

Related U.S. Application Data
(60) Provisional application No. 62/112,942, filed on Feb. 6, 2015.

(51) Int. Cl. C12Q 1/68

(52) U.S. Cl. CPC C12Q 1/6883; C12Q 1/6809; C12Q 2535/122; C12Q 2600/158; C12Q 2600/156

(57) ABSTRACT

Methods and compositions for selecting subjects with a neurological disease such as multiple sclerosis. Also, methods and compositions for identifying poor quality samples, or adulterated samples, as not appropriate for generating a diagnostic result or report.

### CROSS-REFERENCE

This application claims the benefit of U.S. Provisional Application No. 62/112,942, filed Feb. 6, 2015, which application is incorporated herein by reference in its entirety.

### BACKGROUND OF THE INVENTION

Multiple sclerosis (MS) is a demyelinating autoimmune disease of the central nervous system (CNS). MS can be characterized by a wide range of signs and symptoms, including physical, mental, and psychiatric problems. MS can be difficult to diagnose because the symptoms of MS are shared with a number of other diseases. Therefore, there is a tremendous unmet medical need in the diagnosis of Multiple Sclerosis (MS).

False positive results can unnecessarily expose patients who do not have MS to chronic and expensive therapy that, in some cases, actually exacerbates their underlying disease. Alternatively, false negative results can delay those patients who do have MS receiving the correct treatment, which in turn can accelerate the development of permanent physical disability. Problems with sample quality and errors in sample processing can lead to an incorrect, and damaging, diagnostic outcome. Therefore, there is a need in the art for improved quality control processes for use with laboratory testing for MS.

### SUMMARY OF THE INVENTION

A method comprising: (a) amplifying a region comprising two or more codons of a set of variable heavy (VH)4 antibody genes from a nucleic acid sample produced from a subject sample; (b) sequencing the amplified regions using next generation sequencing to generate a set of sequence reads; (c) processing the set of sequence reads to generate a set of (VH)4 sequences; and (d) selecting the subject sample as suitable for diagnostic testing, reporting, or diagnostic testing and reporting when one or more of the following sample quality indicators are met: (i) the set of (VH)4 sequences are from more than a first threshold number of (VH)4 genes, (ii) the set of (VH)4 sequences are from a second threshold number to the first threshold number of (VH)4 antibody genes, and a diversity index for the set of (VH)4 sequences is greater than a diversity index threshold, wherein the second threshold number is less than the first threshold number, (iii) greater than or equal to a first threshold percentage of the set of sequence reads are (VH)4 sequences, (iv) less than or equal to a second threshold percentage of the set of sequence reads contain a CDR3 sequence identical to another sample, or (v) a composite signature score for the set of (VH)4 sequences is not an indeterminate result.

In some embodiments, the subject sample is selected when the set of (VH)4 sequences are from more than the first threshold number of (VH)4 genes.

In some embodiments, wherein the subject sample is selected when the set of (VH)4 gene sequences are from the second threshold number to the first threshold number of (VH)4 genes, and the diversity index for the set of (VH)4 sequences is greater than the diversity index threshold.

In some embodiments, the subject sample is selected when greater than or equal to the first threshold percentage of the set of sequence reads are (VH)4 sequences.

In some embodiments, the subject sample is selected when less than or equal to the second threshold percentage of the set of sequence reads contain a CDR3 sequence identical to another sample.

In some embodiments, the subject sample is selected when the composite signature score for the set of (VH)4 sequences is not an indeterminate result.

In some embodiments, the subject sample is selected when two or more of the sample quality indicators are met.

In some embodiments, the subject sample is selected when three or more of the sample quality indicators are met.

In some embodiments, the subject sample is selected when four of the sample quality indicators are met.

In some embodiments, the first threshold number of (VH)4 genes is about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In some embodiments, the first threshold number of (VH)4 genes is about 30.

In some embodiments, the first threshold number of (VH)4 genes is about: 10-50, 20-40, or 25-35.

In some embodiments, the first threshold number of (VH)4 genes is about: 25-35.

In some embodiments, the second threshold number of (VH)4 genes is about: 1, 2, 3, 4, 5, 6, 7, 8, or 9.

In some embodiments, the second threshold number of (VH)4 genes is about 5.

In some embodiments, the second threshold number of (VH)4 genes is about: 1-9, 2-8, 3-7, or 4-6.

In some embodiments, the second threshold number of (VH)4 genes is about 4-6.

In some embodiments, the diversity index (H′) is calculated using the following formula:

$${H}^{′} = - {\sum }_{i = 1}^{R} {p}_{i} ln {p}_{i}$$

wherein pi is the proportion of the total number of VH4 sequences within a given VH4 antibody subfamily, and R is the total number of species in the subfamily.

In some embodiments, the diversity index threshold is about: 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

In some embodiments, the diversity index threshold is about 1.0.

In some embodiments, the diversity index threshold is about: 1.0-5.0, 1.0-4.0, 1.0-3.0, 1.0-2.5, 1.0-2.0, or 1.0-1.5.

In some embodiments, the diversity index threshold is about 0.85-1.15.

In some embodiments, the first threshold percentage is about: 5%, 10%, 15%, 20%, 25%, 30%, 35%, 40%, 45%, 50%, 55%, 60%, 65%, 70%, 75%, 80%, 85%, 90%, 95% or 99%.

In some embodiments, the first threshold percentage is about 55%.

In some embodiments, the first threshold percentage is about 60%.

In some embodiments, the first threshold percentage is about: 5-99%, 10-95%, 15-90%, 20-85%, 25-80%, 30-75%, 35-70%, 40-65%, 45-60%, or 50-60%.

In some embodiments, the first threshold percentage is about 40-65%.

In some embodiments, the first threshold percentage is about 50-60%.

In some embodiments, the second threshold percentage is about: 5%, 10%, 15%, 20%, 25%, 30%, 35%, 40%, 45%, 50%, 55%, 60%, 65%, 70%, 75%, 80%, 85%, 90%, 95% or 99%.

In some embodiments, the second threshold percentage is about 50%.

In some embodiments, the second threshold percentage is about: 5-99%, 5-95%, 10-90%, 15-85%, 20-80%, 25-75%, 30-70%, 40-60%, or 45-55%.

In some embodiments, the second threshold percentage is about 40-60%.

In some embodiments, the second threshold percentage is about 45-55%.

In some embodiments, the composite signature score is the sum of replacement mutation frequencies at two or more codon positions.

In some embodiments, the replacement mutation frequencies at individual codon positions are normalized by subtracting the average replacement mutation frequency at the same codon position from a healthy control population and dividing by the standard deviation of the average mutation frequency for the same codon position in the healthy control population.

In some embodiments, the two or more codon positions are selected from the group consisting of 31B, 40, 56, 57, 81, and 89.

In some embodiments, the two or more codon positions are selected from the group consisting of 31B, 40, 56, 57, and 81.

In some embodiments, the two or more codon positions are 31B, 40, 56, 57, and 81.

In some embodiments, the indeterminate result is a composite signature score of about 0.8-10.8, 1.8-9.8, 2.8-8.8, 3.8-7.8, 4.8-6.8, 0.8-12.8, 1.8-11.8, 2.8-10.8, 3.8-9.8, 4.8-8.8, or 5.8-7.8.

In some embodiments, the indeterminate result is a composite signature score of about 4.8-6.8.

In some embodiments, the indeterminate result is a composite signature score of about 5.8-7.8.

In some embodiments, the sample comprises beta cells.

In some embodiments, the sample comprises cerebral spinal fluid, blood, or a combination thereof.

In some embodiments, the sample comprises cerebral spinal fluid.

In some embodiments, the sample comprises a cell pellet from cerebral spinal fluid.

In some embodiments, the sample is from a subject suspected of having, or being at risk of developing, a neurological disorder.

In some embodiments, the sample is from a subject suspected of having, or being at risk of developing, multiple sclerosis.

In some embodiments, the multiple sclerosis is relapsing-remitting multiple sclerosis.

In some embodiments, the nucleic acid sample comprises DNA, RNA or a combination thereof.

In some embodiments, the nucleic acid sample comprises genomic DNA, mRNA, cDNA, or a combination thereof.

In some embodiments, the nucleic acid sample comprises genomic DNA.

In some embodiments, the nucleic acid sample comprises whole genome amplified DNA.

In some embodiments, the amplifying comprises specifically hybridizing primers to the region.

In some embodiments, the region comprises codons 24 to 95 of the set of variable heavy (VH)4 antibody genes.

In some embodiments, the region comprises codons 31 to 91 of the set of variable heavy (VH)4 antibody genes.

In some embodiments, next generation sequences comprises 454 sequencing, pyrosequencing, SOLid sequencing, SOLEXA sequencing, SMRT sequencing, nanopore sequencing, ion semiconductor sequencing, DNA nonoball sequencing, or tSMS sequencing.

In some embodiments, next generation sequences comprises 454 sequencing.

In some embodiments, processing the set of individual sequence reads comprises trimming sequences to remove primer sequences, trimming sequences to remove sample barcode sequences, aligning individual sequence reads to each other to identify unique sequences, aligning unique sequences to germline gene segment sequences, removing low quality sequences, removing sequences containing CDR3 sequences that align to a CDR3 sequence from another sample, or a combination thereof.

In some embodiments, processing the set of individual sequence reads comprises aligning unique sequences to germline gene segment sequences.

Some embodiments further comprise, if the sample is selected as suitable for diagnostic testing and reporting, providing a report to a party comprising one or more of: a composite signature score, a diagnosis of having or not having MS, the results of one or more other laboratory tests, or a combination thereof.

In some embodiments, the one or more other laboratory tests include an oligoclonal banding test, an MRI result or image, or a combination thereof.

In some embodiments, the report is provided via a communication medium.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. VH4 and JH gene distributions of CSF B cells from RRMS patients are more divergent from healthy control naive peripheral B cell repertoires than those from OND patients. VH4 (a) and JH (b) gene calls were obtained by IgBlast alignment. Total unique sequences used in cohort databases are indicated inside the pie charts. Statistically significant differences between the frequencies of each cohort were identified by Chi-squared test using a representative pool of 100 sequences from each. Abbreviations: RRMS, relapsing-remitting MS; OND, other neurological disorder; HCN, healthy control naive peripheral B cells.

FIG. 2. Mutation characteristics of VH4 sequences in RRMS and OND patients. (a) Mutation frequency (MF) analysis was done by nucleotide; boxes indicate total unique sequences in each cohort and sample numbers are marked under cohort names. (b) Replacement mutation frequency (RMF) analysis was done by codon. RRMS sequence data includes 119,483 total point mutations and 62,749 total replacement mutations (RM); OND sequence data includes 74,769 total point mutations and 39,324 total replacement mutations (RM); RRMS sequence data includes 51,238 total point mutations and 17,375 total replacement mutations (RM). MF and RMF were calculated by sample and bar graphs show median (indicated on the bar graphs) and interquartile range (statistical significance of the difference between RRMS and OND was tested by Mann Whitney test). MF, RMF and R:S ratios for CDR and FR regions were calculated independently by region for each sample and are shown as cohort medians.

FIG. 3. Antibody Gene Signatures (AGS) in RRMS and OND patients. Each data point represents a single sample sequence pool (median and interquartile range are marked on the figure). The dashed line represents the AGS cut-off point of 6.8 above which patients are expected to have or convert to relapsing-remitting MS (RRMS). The dotted line delineate an indeterminate range (−1) below the 6.8 cut-off where the results of the AGS score test are less clear cut. Samples are grouped by most current diagnosis as RRMS, other neurological diseases (OND), and healthy control naive (HCN). Only samples that pass our filtering criteria are displayed with their calculated AGS scores. Statistical significance of the difference between cohorts was calculated by Mann Whitney test.

FIG. 4. Low diversity correlates with high AGS in the RRMS cohort but not in the OND cohort. Each data point represents a single sample sequence pool from (a) the RRMS cohort or (b) the OND cohort. The diversity index was calculated as described in the methods section and high values indicate a more even distribution across the VH4 genes. Pearson's correlation coefficient (R) indicates the linear correlation between AGS and the diversity index, and the two-tailed p-value of the correlation is also indicated. The dashed line represents the AGS cut-off point of 6.8 above which patients are expected to have or convert to relapsing-remitting MS (RRMS). The dotted lines delineate an indeterminate range (−1) below the 6.8 cut-off where the results of the AGS score test are less clear cut. (c) Distribution of the diversity index is shown here with the median marked on the graph. HCN has one point per replicate. Statistical significance of the difference between cohorts was tested by Mann Whitney test.

FIG. 5. AGS does not correlate with age, MF % or RMF % in both RRMS and OND. Each data point represents a single sample sequence pool. Pearson's correlation coefficient (r) indicates the linear correlation between AGS and either age in years, mutation frequency (MF %), or replacement mutation frequency (RMF %). The two-tailed p-value of the correlation is also indicated.

FIG. 6. Diversity index does not correlate with sequence number in both RRMS and OND. Each data point represents a single sample sequence pool. Two high sequence number outliers were removed because they had more than the median+2 standard deviations of the sequences of all CSF samples (>1,431 unique sequences). Pearson's correlation coefficient (r) indicates the linear correlation between the diversity index and the number of unique sequences in the sample. The two-tailed p-value of the correlation is also indicated.

FIG. 7. Antibody Gene Signature (AGS) in all RRMS and OND patients. Each data point represents a single sample sequence pool (median and interquartile range are marked on the figure). The dashed line represents the AGS cut-off point of 6.8 above which patients are expected to have or convert to relapsing-remitting MS (RRMS). The dotted lines delineate an indeterminate range (−1) below the 6.8 cut-off where the results of the AGS score test are less clear cut. Samples are grouped by most current diagnosis as RRMS, other neurological diseases (OND), and healthy control naive (HCN). OND samples that were filtered out due to low sequence count are added to with an assigned AGS score of −8.9 (minimum score). Statistical significance of the difference between cohorts was tested by Mann Whitney test.

FIG. 8. Exemplary process overview chart for sequencing VH(4) codons from subject samples.

FIG. 9. Exemplary process overview chart for sequencing VH(4) codons from cerebral spinal fluid (CSF) cell pellet samples using next generation sequencing.

FIG. 10. Exemplary process overview chart for isolation of genomic DNA (gDNA) from a CSF cell pellet.

FIG. 11. Exemplary process overview chart for performing whole genome amplification (WGA) and subsequent clean-up on genomic DNA (gDNA).

FIG. 12. Exemplary process overview chart for performing target specific amplification of immunoglobulin heavy chain variable (VH)-diversity (DH)-joining (JH) (VDJ) regions from WGA DNA.

FIG. 13. Exemplary process overview chart for barcoding and sequencing VDJ amplicons.

FIG. 14. A computer system useful for displaying, storing, retrieving, transmitting or calculating results from the analysis of subject samples (e.g., a VH4 codon signature); displaying, storing, retrieving, or calculating raw data from (VH)4 VDJ sequence analysis; or displaying, storing, retrieving, or calculating any sample or subject information useful in the methods disclosed herein.

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

### Definitions

Unless characterized differently, technical and scientific terms used herein have the same meaning as commonly understood by one of ordinary skill in the art.

The term “about” in relation to a reference numerical value includes a range of values plus or minus 10% from that value. All numerical indications in this specification are to be understood as being qualified by the term “about,” unless the context clearly indicates otherwise.

The term “or” can be used conjunctively or disjunctively.

The term “diagnosis” as used herein and its grammatical equivalents, means the testing of subjects to determine if they have a particular trait for use in a clinical decision. Diagnosis includes testing of subjects at risk of developing a particular disease resulting from infection by an infectious organism or a non-infectious disease, such as cancer or a metabolic disease. The disease can be multiple sclerosis (e.g., relapsing and remitting multiple sclerosis). Diagnosis also includes testing of subjects who have developed particular symptoms to determine the cause of the symptoms. Diagnosis also includes prognosis, monitoring progress of a disease, and monitoring the efficacy of therapeutic regimens. The result of a diagnosis can be used to classify patients into groups for performance of clinical trials for administration of certain therapies.

“Patient” or “subject” includes mammals, such as humans, including those in need of treatment thereof. Humans can include, e.g., babies, children, teenagers, adults, and the elderly.

### Methods of the Invention

Introduction

Multiple sclerosis (MS) is a demyelinating autoimmune disease of the central nervous system (CNS). In MS, damage to the myelin sheath of neurons in the brain and spinal cord disrupts the ability of those neurons to communicate. MS can be characterized by a wide range of signs and symptoms, including physical, mental, and psychiatric problems. MS can take several forms, with new symptoms either occurring in isolated attacks (relapsing-remitting MS or RRMS) or building up over time (primary-progressive MS or PPMS).

The symptoms of MS can be variable and unpredictable, and can vary depending upon which part of the nervous system is damaged. More common can symptoms include fatigue, numbness or tingling, weakness, dizziness and vertigo, sexual problems, pain, emotional changes, walking (gait) difficulties, spasticity, vision problems, bladder problems, bowel problems, cognitive changes, and depression. Less common symptoms can include speech problems, tremors, breathing problems, headache, swallowing problems, seizures, itching, and hearing loss. Because MS symptoms can overlap with other medical conditions, diagnosis of MS can be difficult, especially in the early stages of RRMS.

A (VH)4 codon signature has been identified to aid in the diagnosis of MS (e.g., RRMS). One aspect of the present disclosure are methods wherein this (VH)4 codon signature is detected in samples from a subject or patient. The methods can include techniques for the isolation of nucleic acids from the subject sample, non-specific amplification of nucleic acids, target specific amplification of nucleic acids, sequencing (e.g., next generation sequencing) of nucleic acids, or any combination there. The methods disclosed herein can be used in a laboratory test to aid in the diagnosis of MS (e.g., RRMS).

The reliability of a laboratory test (e.g., a genetic test) may be affected by a number of factors during the sample collection phase, the sample processing phase, and the analytical phase. For example, samples can be mislabeled, samples can become contaminated, insufficient material can be collected, sample deterioration can occur during transport, incorrect testing procedures can be used, etc.

In addition, tests that use nucleotide sequencing techniques (e.g., next generation sequencing) introduce additional challenges because these techniques can suffer from sequencing errors. Next generation sequencing techniques generate huge amounts of data and each of the techniques can have built in biases or error profiles. In addition, many sequencing protocols and sample preparation protocols utilize one or more PCR based amplification steps. These amplifications, while allowing for the processing of samples containing low levels of the subject nucleotides, can also amplify replication errors and small amounts of cross-contamination. Indeed, the differentiation of true variation and context-specific sequencing errors is a major challenge in next generation sequencing (NGS) analysis.

An incorrect test result may lead to: unnecessary and irreversible interventions, which may in themselves have associated risks for the patient, inaccurate risk assessment regarding the disease, and missed opportunities for disease prevention or treatment. There is a need in the art to develop diagnostic methods that reduce the risks associated with incorrect test results.

Accordingly, another aspect of the present disclosure are methods for determining whether a sample is of sufficient quality to, for example, generate an accurate or reportable VH4 codon signature or diagnosis. These methods can detect samples containing insufficient materials (e.g., B cells) or samples that have become contaminated.

VH4 Codon Signature for Multiple Sclerosis

Multiple sclerosis (MS) is a demyelinating autoimmune disease of the central nervous system (CNS). Several studies have underscored the impact of T and B cells in this disease and have broadened the community's search for more effective immunomodulatory therapies for the treatment of MS. For example, early evidence for a role of B cells in the pathoetiology of MS, including oligoclonal bands, altered antibody genetics and B cell responses to neuroantigens in vitro provided the basis for use of Rituximab, a B cell depleting antibody for the efficacious treatment of MS.

B cells in the CNS of MS patients can undergo extensive clonal expansion, and in some cases, can recognize neuroantigens. Because antigen-driven B cell selection can be dependent on somatic hypermutation (SHM) accumulation, the CSF-derived B cell pool of MS patients may be enriched for a unique pattern of SHM that would reflect their potential to recognize neuroantigens. It is demonstrated and confirmed herein that CSF-derived B cells from MS patients expressing rearranged variable heavy chain family 4 (VH4) genes accumulate replacement mutations at 6 codon positions.

VH4

The immune system can generate millions of antibodies with different antigen binding abilities. The diversity is brought about by the complexities of constructing immunoglobulin molecules. These molecules contain paired polypeptide chains (heavy and light), with each containing a constant and a variable region. The structures of the variable regions of the heavy and light chains are specified by immunoglobulin V genes. The heavy chain variable region is derived from three gene segments known as VH, D and JH. In humans, there can be at least 100 different VH segments, over 20 D segments and six JH segments. The light chain genes have two segments: the VL and JL segments. Antibody diversity can result from random combinations of VH/D/JH (VDJ) segments with VLJL components superimposed on which are several mechanisms including junctional diversity and somatic mutation.

The germline VH genes can be separated into at least six families (VH1 through VH6) based on DNA nucleotide sequence identity of the first 95 to 101 amino acids. Members of the same family typically have ≧80% sequence identity, whereas members of different families typically have less than 70% identity. These families range in size from one VH6 gene to an estimated greater than 45 VH3 genes. In addition, many pseudogenes exist. Recent studies have nearly completed a physical map of the VH locus on chromosome 14q32.13.15. It has now been estimated that the human VH repertoire is represented by approximately 50 functional VH segments with about an equal number of pseudogenes. These studies estimate the size of the VH locus to be approximately 1100 kb, which is less than half the previous estimates of 2.5 to 3 megabases as determined by pulse field gel electrophoresis. The VH4 family of genes contains 9 different members: 4-04, 4-28, 4-30, 4-31, 4-34, 4-39, 4-59, 4-61, 4-B4.

A “signature” in the VH4 sequences of certain B cells has been identified as associated with multiple sclerosis (see, U.S. Pat. No. 8,394,583, which is hereby incorporated by reference in its entirety). The sequence signature can comprise one or more codons from, for example, codons 24 to 95 of a set of VH4 genes. The sequence signature can comprise one or more of codons 31B, 40, 56, 57, 81, or 89. The sequence signature can comprise codons 31B, 40, 56, 57, and 81. The sequence signature can comprise codons 31B, 40, 56, 57, 81, and 89. By examining the sequence at these positions, and identifying mutations or mutation frequencies at one or more of the codon positions, a subject can be identified as being at risk of developing or having multiple sclerosis.

Sample Collection

One or more biological samples can be collected from a subject for analysis. In some embodiments, the one or more biological samples comprise a blood sample, a cerebral spinal fluid sample, a tissue sample, or a combination thereof. Any sample that contains B cells can be used.

A tissue sample can be collected, for example, by needle biopsy, punch biopsy, surgical biopsy, or a combination thereof. The biopsy can be a guided biopsy (e.g., CT-guided biopsy, ultrasound-guided biopsy).

A blood sample can be collected, for example, by venipuncture, of finger sticking. Blood samples can be collected, for example, in a tube (e.g., a vacuum tube, a capillary tube), a syringe, or a bag.

A cerebral spinal fluid (CSF) sample can be collected, for example, by a lumbar puncture or spinal tap. CSF samples can be collected, for example, in a tube (e.g., a vacuum tube, a capillary tube), a syringe, or a bag.

Blood and CSF samples, or cell pellets produced after centrifugation of the blood or CSF sample, can be frozen and shipped to another site for processing. Alternatively, the samples can be processed on site.

In some instances, samples may be collected from individuals repeatedly over a longitudinal period of time (e.g., once a day, once a week, once a month, biannually or annually). Obtaining numerous samples from an individual over a period of time can be used to verify results from earlier detections and/or to identify an alteration as a result of, for example, drug treatment. Samples can be obtained from humans or non-humans.

In this disclosure, biological samples from a subject or patient can be referred to as subject samples.

Isolation of Nucleic Acids

An aspect of the present disclosure concerns isolation of DNA segments and their use in detecting the presence of mutations in certain codons of the VH4 segments from a subject. Many methods described herein will involve the use of amplification primers, oligonucleotide probes, and other nucleic acid elements involved in the analysis of genomic DNA, cDNA or mRNA transcripts, such as the germline or normal sequence of VH4 family genes.

A “nucleic acid” as used herein will generally refer to a molecule (i.e., a strand) of DNA or RNA comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product.

These definitions generally refer to a single-stranded molecule, but can also encompass an additional strand that is partially, substantially or fully complementary to the single-stranded molecule. Thus, a nucleic acid may encompass a double-stranded molecule that comprises complementary strands or “complements” of a particular sequence comprising a molecule. In particular aspects, a nucleic acid encodes a protein or polypeptide, or a portion thereof.

The methods disclosed herein can include producing nucleic acid samples from subject samples. Methods of isolating nucleic acids (e.g., DNA, e.g., genomic DNA; RNA, e.g., messenger RNA) are known and any suitable method may be used. Additionally, commercially available kits can be used.

Amplification of Nucleic Acids

Nucleic acids used as a template for amplification may be isolated from cells, tissues or other samples according to standard methodologies. Alternatively, analysis can be performed on whole cell or tissue homogenates or biological fluid samples with or without substantial purification of the template nucleic acid. The nucleic acid may be DNA (e.g., genomic DNA) and can be fractionated or RNA (e.g., whole cell RNA, messenger RNA, etc.). Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred. Primers can include barcode sequences, adaptor sequences, universal sequencing sequences, or a combination thereof. Barcode sequences can be used to tag all nucleic acids isolated from a single sample. Barcode sequences can also provide a unique sequence tag for each nucleic acid in a nucleic acid sample.

Non-Specific Amplification

Nucleic acids (e.g., genomic DNA) can be non-specifically amplified, for example, to increase the amount of the nucleic acids available for analysis. Where the nucleic acids comprise genomic DNA, whole genome amplification (WGA). A non-specific amplification, such WGA, can use random hexamer primers.

Target Specific Amplification

Pairs of primers designed to selectively hybridize to nucleic acids corresponding to the variable heavy chain gene locus, variants and fragments thereof can be contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

Primers can be degenerate target-specific PCR primers, non-degenerate target-specific PCR primers, or a combination thereof. Primers can be designed based upon an alignment of known sequences (e.g., known (VH)4 sequences). Primers can be designed to selectively hybridize to conserved regions of known sequences (e.g., conserved regions of a set of (VH)4 sequences.

Primers can be designed to amplify two or more codons of a set of variable heavy (VH)4 antibody genes. For example, the two or more codon positions can be selected from codons 24 to 95, or from codons 31 to 92. In another example, the two or more codon positions can be selected from the group consisting of 31B, 40, 56, 57, 81, and 89. In another example, the two or more codons can be selected from the group consisting of 31B, 40, 56, 57, and 81. In another example, the two or more codons can include 31B, 40, 56, 57, and 81.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Target specific amplification can be performed, for example, in a two-step nested PCR reaction, or a single step PCR reaction. In some embodiments, a two-step nested PCR protocol and degenerate target-specific PCR primers is used. In some embodiments, a two-step nested PCR protocol using pools of non-degenerate PCR target-specific PCR primers is used. In some embodiments, a single PCR reaction incorporating a pool of non-degenerate target-specific PCR primers is used.

Primer extension, which may be used as a stand-alone technique or in combination with other methods (such as PCR), requires a labeled primer (usually 20-50 nucleotides in length) which is complementary to a region near the 5′ end of the gene. The primer is allowed to anneal to the RNA and reverse transcriptase is used to synthesize complementary cDNA to the RNA until it reaches the 5′ end of the RNA.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Real-time polymerase chain reaction, also called quantitative real time polymerase chain reaction (qPCR) or kinetic polymerase chain reaction, is a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA.

Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type. Although real-time quantitative polymerase chain reaction is often marketed as RT-PCR, it should not be confused with reverse transcription polymerase chain reaction, also known as RT-PCR.

A DNA-binding dye binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR Green will bind to all dsDNA PCR products, including non-specific PCR products (such as “primer dimers”). This can potentially interfere with or prevent accurate quantification of the intended target sequence. The reaction is prepared as usual, with the addition of fluorescent dsDNA dye.

The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained may not have absolute units associated with it (i.e. mRNA copies/cell). As described above, a comparison of a measured DNA/RNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene. This can correct possible differences in RNA quantity or quality across experimental samples.

Using fluorescent reporter probes can be an accurate reliable method, but can also be expensive. It uses a sequence-specific RNA or DNA-based probe to quantify only the DNA containing the probe sequence; therefore, use of the reporter probe significantly increases specificity, and allows quantification even in the presence of some non-specific DNA amplification. This potentially allows for multiplexing—assaying for several genes in the same reaction by using specific probes with different-coloured labels, provided that all genes are amplified with similar efficiency.

It is commonly carried out with an RNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter.

The PCR reaction can be prepared as usual (see PCR), and the reporter probe is added. As the reaction commences, during the annealing stage of the PCR both probe and primers anneal to the DNA target. Polymerization of a new DNA strand is initiated from the primers, and once the polymerase reaches the probe, its 5′-3-exonuclease degrades the probe, physically separating the fluorescent reporter from the quencher, resulting in an increase in fluorescence.

Fluorescence is detected and measured in the real-time PCR thermocycler, and its geometric increase corresponding to exponential increase of the product is used to determine the threshold cycle (CT) in each reaction.

Quantitating gene expression by traditional methods presents several problems. Firstly, detection of mRNA on a Northern blot or PCR products on a gel or Southern blot is time-consuming and does not allow precise quantitation. Also, over the 20-40 cycles of a typical PCR reaction, the amount of product reaches a plateau determined more by the amount of primers in the reaction mix than by the input template/sample.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Ct. Since the quantity of DNA doubles every cycle during the exponential phase, relative amounts of DNA can be calculated, e.g., a sample whose Ct is 3 cycles earlier than another's has 23=8 times more template.

Amounts of RNA or DNA are then determined by comparing the results to a standard curve produced by RT-PCR of serial dilutions (e.g., undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Choosing a reference gene fulfilling this criterion is therefore of high importance, and often challenging, because only very few genes show equal levels of expression across a range of different conditions or tissues.

Cleaning Nucleic Acids

Following any amplification, it may be desirable to clean the product, for examply, by separating the amplification product from the template and/or the excess primer. Amplification products are separated by agarose, agarose-acrylamide or polyacrylamide gel electrophoresis using standard methods. Separated amplification products may be cut out and eluted from the gel for further manipulation. Using low melting point agarose gels, the separated band may be removed by heating the gel, followed by extraction of the nucleic acid.

Cleaning of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC. Commercially available kits can be used to clean the nucleic acids.

Detection/Quantification of Nucleic Acids

Nucleic acids (e.g., amplification products) can be visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

Nucleic acids (e.g., amplification products) can be quantitated using spectroscopy. Additionally, commercially available kits can be used to detect and or quantitate nucleic acids (e.g., amplification products).

DNA Sequencing

Nucleic acids (e.g., an amplified region of a variable heavy (VH)4 antibody gene) can be sequenced. Sequencing can be accomplished using either the “dideoxy-mediated chain termination method,” also known as the “Sanger Method” or the “chemical degradation method,” also known as the “Maxam-Gilbert method.” Sequencing can be performed by next generation sequencing.

Next Generation Sequencing

The methods described herein can use one or more next-generation sequencing techniques to determine the status of one or more molecular markers in a sample from a subject (e.g., the sequence of a set of variable heavy (VH)4 antibody genes. Next-generation sequencing techniques include, for example, Helicos True Single Molecule Sequencing (tSMS) (Harris T. D. et al. (2008) Science 320:106-109); 454 sequencing (Roche) (Margulies, M. et al. 2005, Nature, 437, 376-380); SOLiD technology (Applied Biosystems); SOLEXA sequencing (Illumina); single molecule, real-time (SMRT™) technology of Pacific Biosciences; nanopore sequencing (Soni G V and Meller A. (2007) Clin Chem 53: 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g. Oxford Nanopore, Genia Technologies, and Nabsys).

The next generation sequencing technique can be 454 sequencing (Roche) (see e.g., Margulies, M et al. (2005) Nature 437: 376-380). 454 sequencing can involve two steps. In the first step, DNA can be sheared into fragments of approximately 300-800 base pairs, and the fragments can be blunt ended. Oligonucleotide adaptors can then ligated to the ends of the fragments. The adaptors can serve as sites for hybridizing primers for amplification and sequencing of the fragments. The fragments can be attached to DNA capture beads, e.g., streptavidin-coated beads using, e.g., Adaptor B, which can contain 5′-biotin tag. The fragments can be attached to DNA capture beads through hybridization. A single fragment can be captured per bead. The fragments attached to the beads can be PCR amplified within droplets of an oil-water emulsion. The result can be multiple copies of clonally amplified DNA fragments on each bead. The emulsion can be broken while the amplified fragments remain bound to their specific beads. In a second step, the beads can be captured in wells (pico-liter sized; PicoTiterPlate (PTP) device). The surface can be designed so that only one bead fits per well. The PTP device can be loaded into an instrument for sequencing. Pyrosequencing can be performed on each DNA fragment in parallel. Addition of one or more nucleotides can generate a light signal that can be recorded by a CCD camera in a sequencing instrument. The signal strength can be proportional to the number of nucleotides incorporated.

Pyrosequencing can make use of pyrophosphate (PPi) which can be released upon nucleotide addition. PPi can be converted to ATP by ATP sulfurylase in the presence of adenosine 5′ phosphosulfate. Luciferase can use ATP to convert luciferin to oxyluciferin, and this reaction can generate light that can be detected and analyzed. The 454 Sequencing system used can be GS FLX+ system or the GS Junior System.

The next generation sequencing technique can be SOLiD technology (Applied Biosystems; Life Technologies). In SOLiD sequencing, genomic DNA can be sheared into fragments, and adaptors can be attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured and beads can be enriched to separate the beads with extended templates. Templates on the selected beads can be subjected to a 3′ modification that permits bonding to a glass slide. A sequencing primer can bind to adaptor sequence. A set of four fluorescently labeled di-base probes can compete for ligation to the sequencing primer. Specificity of the di-base probe can be achieved by interrogating every first and second base in each ligation reaction. The sequence of a template can be determined by sequential hybridization and ligation of partially random oligonucleotides with a determined base (or pair of bases) that can be identified by a specific fluorophore. After a color is recorded, the ligated oligonucleotide can be cleaved and removed and the process can be then repeated. Following a series of ligation cycles, the extension product can be removed and the template can be reset with a primer complementary to the n−1 position for a second round of ligation cycles. Five rounds of primer reset can be completed for each sequence tag. Through the primer reset process, most of the bases can be interrogated in two independent ligation reactions by two different primers. Up to 99.99% accuracy can be achieved by sequencing with an additional primer using a multi-base encoding scheme.

The next generation sequencing technique can be SOLEXA sequencing (ILLUMINA sequencing). ILLUMINA sequencing can be based on the amplification of DNA on a solid surface using fold-back PCR and anchored primers. ILLUMINA sequencing can involve a library preparation step. Genomic DNA can be fragmented, and sheared ends can be repaired and adenylated. Adaptors can be added to the 5′ and 3′ ends of the fragments. The fragments can be size selected and purified. ILLUMINA sequence can comprise a cluster generation step. DNA fragments can be attached to the surface of flow cell channels by hybridizing to a lawn of oligonucleotides attached to the surface of the flow cell channel. The fragments can be extended and clonally amplified through bridge amplification to generate unique clusters. The fragments become double stranded, and the double stranded molecules can be denatured. Multiple cycles of the solid-phase amplification followed by denaturation can create several million clusters of approximately 1,000 copies of single-stranded DNA molecules of the same template in each channel of the flow cell. Reverse strands can be cleaved and washed away. Ends can be blocked, and primers can by hybridized to DNA templates. ILLUMINA sequencing can comprise a sequencing step. Hundreds of millions of clusters can be sequenced simultaneously. Primers, DNA polymerase and four fluorophore-labeled, reversibly terminating nucleotides can be used to perform sequential sequencing. All four bases can compete with each other for the template. After nucleotide incorporation, a laser can be used to excite the fluorophores, and an image is captured and the identity of the first base is recorded. The 3′ terminators and fluorophores from each incorporated base are removed and the incorporation, detection and identification steps are repeated. A single base can be read each cycle. In some embodiments, a HiSeq system (e.g., HiSeq 2500, HiSeq 1500, HiSeq 2000, or HiSeq 1000) is used for sequencing. In some embodiments, a MiSeq personal sequencer is used. In some embodiments, a Genome Analyzer IIx is used.

The next generation sequencing technique can be real-time (SMRT™) technology by Pacific Biosciences. In SMRT, each of four DNA bases can be attached to one of four different fluorescent dyes. These dyes can be phospholinked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zeptoliters (10˜21 liters) can be created. The tiny detection volume can provide 1000-fold improvement in the reduction of background noise. Detection of the corresponding fluorescence of the dye can indicate which base was incorporated. The process can be repeated.

The next generation sequencing can be nanopore sequencing (See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than about 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or S1O2). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, nanopore sequencing technology from GENIA is used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some embodiments, an IONPROTON™ Sequencer is used to sequence nucleic acid. In some embodiments, an IONPGM™ Sequencer is used.

The next generation sequencing can be DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template. Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

The next generation sequencing technique can be Helicos True Single Molecule Sequencing (tSMS) (see e.g., Harris T. D. et al. (2008) Science 320:106-109). In the tSMS technique, a DNA sample can be cleaved into strands of approximately 100 to 200 nucleotides, and a polyA sequence can be added to the 3′ end of each DNA strand. Each strand can be labeled by the addition of a fluorescently labeled adenosine nucleotide. The DNA strands can then be hybridized to a flow cell, which can contain millions of oligo-T capture sites immobilized to the flow cell surface. The templates can be at a density of about 100 million templates/cm². The flow cell can then be loaded into an instrument, e.g., HELISCOPE™ sequencer, and a laser can illuminate the surface of the flow cell, revealing the position of each template. A CCD camera can map the position of the templates on the flow cell surface. The template fluorescent label can then be cleaved and washed away. The sequencing reaction can begin by introducing a DNA polymerase and a fluorescently labeled nucleotide. The oligo-T nucleic acid can serve as a primer. The DNA polymerase can incorporate the labeled nucleotides to the primer in a template directed manner. The DNA polymerase and unincorporated nucleotides can be removed. The templates that have directed incorporation of the fluorescently labeled nucleotide can be detected by imaging the flow cell surface. After imaging, a cleavage step can remove the fluorescent label, and the process can be repeated with other fluorescently labeled nucleotides until a desired read length is achieved. Sequence information can be collected with each nucleotide addition step. The sequencing can be asynchronous. The sequencing can comprise at least 1 billion bases per day or per hour.

The sequencing technique can comprise paired-end sequencing in which both the forward and reverse template strand can be sequenced. In some embodiments, the sequencing technique can comprise mate pair library sequencing. In mate pair library sequencing, DNA can be fragments, and 2-5 kb fragments can be end-repaired (e.g., with biotin labeled dNTPs). The DNA fragments can be circularized, and non-circularized DNA can be removed by digestion. Circular DNA can be fragmented and purified (e.g., using the biotin labels). Purified fragments can be end-repaired and ligated to sequencing adaptors.

Processing of Next Generation Sequencing Data

Raw sequence reads from next generation sequencing can be processed and analyzed using computer analysis tools (e.g., the VDJserver online repertoire analysis tool).

Reads can be trimmed of all primers and sample barcode sequences, and then aligned to each other within a sample. The alignment can allow for edge mismatching. For example, the alignment can allow a total edge mismatch of 5 nucleotides between sequences identified as matching. The number of copies of each unique sequence can be noted as a sequence tag for subsequent analysis and filtering.

Each unique sequence can be aligned to germline gene segment sequences. The alignment can be performed, for example, using the IgBlast aligner through VDJserver.

Initial filtering can be performed to remove sequences that are identified as having an error or as being of low quality. Sequences can be removed when a mean sequence quality is less than 35. Sequences can be removed when the length of the sequence is truncated (e.g., shorter than, e.g., 200 nucleotides). Sequences can be removed when one or more sequencing errors, such as frame-shifting insertions or deletions, out of frame junctions, or inappropriate stop codons are present. Sequences can be removed that have less than 85% homology to a germline sequence. Sequences with low representation (e.g., having fewer than two copies) can be removed.

### Predicting and Diagnosing Multiple Sclerosis

Multiple Sclerosis

Multiple Sclerosis (MS) is one of the most common diseases of the central nervous system (brain and spinal cord). It is an inflammatory condition associated with demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord, hence the term “multiple” sclerosis, literally meaning many scars.

There is a tremendous unmet medical need in the diagnosis of Multiple Sclerosis (MS). Patients that present with non-specific clinical symptoms or symptoms that are consistent with MS currently undergo a battery of diagnostic tests.

MS can be difficult to diagnose because the symptoms of MS are shared with a number of other diseases. For example, symptoms of MS can easily be confused with a wide variety of other diseases such as acute disseminated encephalomyelitis, Lyme disease, HIV-associated myelopathy, HTLV-I-associated myelopathy, neurosyphilis, progressive multifocal leukoencephalopathy, systemic lupus erythematosus, polyarteritis nodosa, Sjögren's syndrome, Behcet's disease, sarcoidosis, paraneoplastic syndromes, subacute combined degeneration of cord, subacute myelo-optic neuropathy, adrenomyeloneuropathy, spinocerebellar syndromes, hereditary spastic paraparesis/primary lateral sclerosis, strokes, tumors, arteriovenous malformations, arachnoid cysts, Arnold-Chiari malformations, and cervical spondylosis. Consequently, the diagnosis of MS is generally made by a process that demonstrates findings that are consistent with MS, and also rules out other causes.

The current diagnostic standard for MS includes analysis of cerebral spinal fluid (CSF) using the oligoclonal banding (OCB) test alongside magnetic resonance imaging (MRI) and a comprehensive set of clinical tests to rule-out other neurological diseases. In an OCB test, the presence of unique bands in the cerebral spinal fluid as compared to a peripheral blood sample (e.g., 3 or more unique bands) can be indicative of MS. However, reported overall accuracy of the OCB test is approximately 54-69%. A diagnosis of having MS can require that there have been two attacks at least one month apart. An attack, also known as an exacerbation, flare, or relapse, can be a sudden appearance of or worsening of an MS symptom or symptoms which lasts at least 24 hours. A diagnosis of having MS can require more than one area of damage to central nervous system myelin sheath. Damage to sheath should have occurred at more than one point in time and not have been caused by any other disease that can cause demyelination or similar neurologic symptoms. MRI (magnetic resonance imaging) to image the brain can be used to detect the presence of plaques or scarring caused by MS.

The diagnosis of MS may not be able to be made, however, solely on the basis of MRI. Other diseases can cause comparable lesions in the brain that resemble those caused by MS. Furthermore, the appearance of brain lesions by MRI can be quite heterogeneous in different patients, even resembling brain or spinal cord tumors in some. In addition, a normal MRI scan does not rule out a diagnosis of MS, as a small number of patients with confirmed MS do not show any lesions in the brain on MRI. These individuals often have spinal cord lesions or lesions which cannot be detected by MRI. As a result, a thorough clinical exam can include a patient history and functional testing. The clinical exam can cover mental, emotional, and language functions, movement and coordination, vision, balance, and the functions of the five senses. Sex, birthplace, family history, and age of the person when symptoms first began may also be important considerations. Other tests, including evoked potentials (electrical diagnostic studies that may reveal delays in central nervous system conduction times), cerebrospinal fluid (seeking the presence of clonally-expanded immunoglobulin genes, referred to as oligoclonal bands), and blood (to rule out other causes), may be used.

A (VH)4 codon signature has been identified to aid in the diagnosis of MS (e.g., RRMS). One aspect of the present disclosure are methods wherein this (VH)4 codon signature is detected in samples from a subject or patient. The methods can include techniques for the isolation of nucleic acids from the subject sample, non-specific amplification of nucleic acids, target specific amplification of nucleic acids, sequencing (e.g., next generation sequencing) of nucleic acids, or any combination there. The methods disclosed herein can be used in a laboratory test to aid in the diagnosis of MS (e.g., RRMS).

False positive results can unnecessarily expose patients who do not have MS to chronic and expensive therapy that, in some cases, actually exacerbates their underlying disease. Alternatively, false negative results can delay those patients who do have MS receiving the correct treatment, which in turn can accelerate the development of permanent physical disability. Therefore, an aspect of this disclosure is methods of selecting samples that are of sufficient quality to generate an accurate or reportable VH4 codon signature or diagnosis. These methods can detect samples containing insufficient materials (e.g., B cells) or samples that have become contaminated.

The methods disclosed herein can comprise: (a) amplifying a region comprising two or more codons of a set of variable heavy (VH)4 antibody genes from a nucleic acid sample produced from a subject sample; (b) sequencing the amplified regions using next generation sequencing to generate a set of sequence reads; (c) processing the set of sequence reads to generate a set of (VH)4 sequences; and (d) selecting the subject sample as suitable for diagnostic testing, reporting, or diagnostic testing and reporting when one or more of the following sample quality indicators are met: (i) the set of (VH)4 sequences are from more than a first threshold number of (VH)4 genes, (ii) the set of (VH)4 sequences are from a second threshold number to the first threshold number of (VH)4 antibody genes, and a diversity index for the set of (VH)4 sequences is greater than a diversity index threshold, wherein the second threshold number is less than the first threshold number, (iii) greater than or equal to a first threshold percentage of the set of sequence reads are (VH)4 sequences, (iv) less than or equal to a second threshold percentage of the set of sequence reads contain a CDR3 sequence identical to another sample, or (v) a composite signature score for the set of (VH)4 sequences is not an indeterminate result.

Samples Processing for Sequence Analysis

Included in this disclosure are methods of sample processing for sequencing analysis. The methods of sample processing disclosed herein can be flexible and can allow for a wide range of differing protocols. The methods can comprise (a) amplifying a region comprising two or more codons of a set of variable heavy (VH)4 antibody genes from a nucleic acid sample produced from a subject sample; and (b) sequencing the amplified regions using next generation sequencing to generate a set of sequence reads. The following description outlines exemplary protocols. However, variations on these protocols are contemplated.

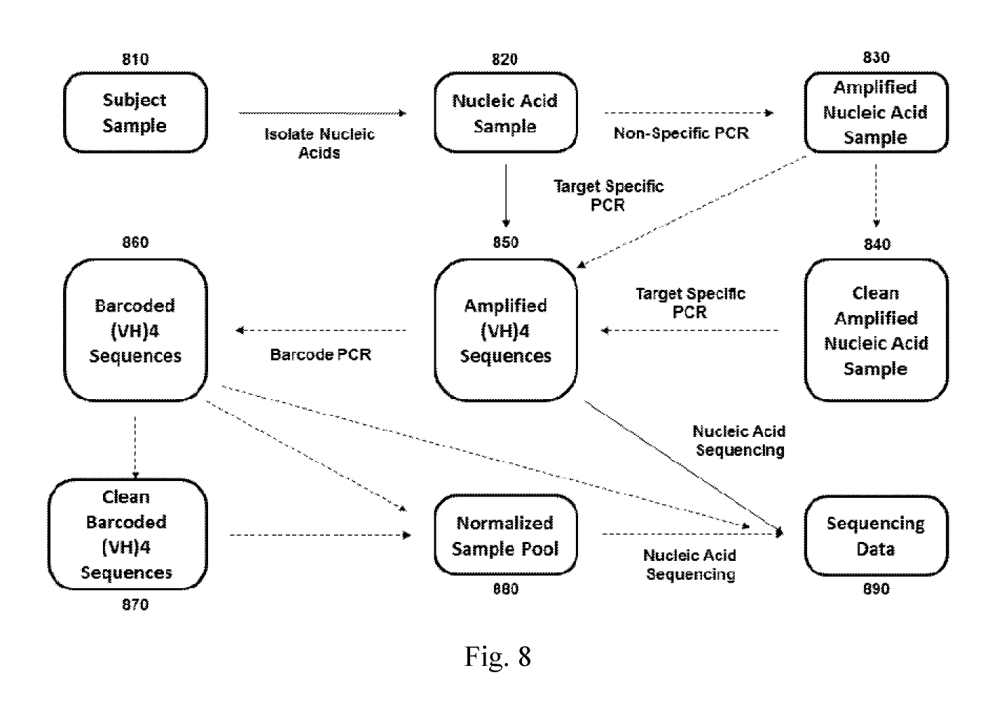

A broad overview of an exemplary method of processing of a subject sample to produce sequencing data of a set of (VH)4 sequences is shown in FIG. 8. Nucleic acids can be isolated from a Subject Sample 810 to produce a Nucleic Acid Sample 820. Target specific PCR can be performed on the Nucleic Acid Sample 820 to produce Amplified (VH)4 Sequences 850. The Amplified (VH)4 Sequences 850 can be sequenced to produce Sequencing Data 890.

The Subject Sample 810 can be any biological sample that contains B cells. For example, the Subject Sample can be a blood sample, a cerebral spinal fluid (CSF) sample, a tissue sample, or a combination thereof. In some embodiments, the Subject Sample can be a blood sample. In some embodiments, the Subject Sample can be a CSF sample. Where the Subject Sample is a blood sample or a CSF sample, the Subject Sample can be a fluid sample containing cells, a cell pellet, or a combination thereof. In some embodiments, the Subject Sample is a CSF cell pellet.

Referring to FIG. 8, nucleic acids can be isolated from the Subject Sample 810 to produce a Nucleic Acid Sample 820. The Nucleic Acid Sample can contain DNA, RNA, or a combination thereof. In some embodiments, the Nucleic Acid Sample contains DNA (e.g., genomic DNA (gDNA)). In some embodiments, the Nucleic Acid Sample contains RNA (e.g., messenger RNA (mRNA)). Any method can be used for the isolation of the nucleic acids. The selection of the method can be determined by the type of nucleic acids desired. Where the Nucleic Acid Sample contains mRNA, cDNA can be produced from the mRNA using a reverse transcription reaction. The amount of nucleic acids can be quantitated.

Referring to FIG. 8, target specific PCR can be performed on the Nucleic Acid Sample 820 to produce Amplified (VH)4 Sequences 850. The PCR reaction can be performed using degenerate target-specific PCR primers, non-degenerate target-specific PCR primers, or a combination thereof. The PCR reaction can be a two-step nested PCR reaction, or a single step PCR reaction. In some embodiments, a two-step nested PCR protocol and degenerate target-specific PCR primers is used. In some embodiments, a two-step nested PCR protocol using pools of non-degenerate PCR target-specific PCR primers is used. In some embodiments, a single PCR reaction incorporating a pool of non-degenerate target-specific PCR primers is used. The PCR Primers can be designed based upon an alignment of known (VH)4 sequences. The PCR primers, or a subset of the PCR primers, used in target specific PCR can include one or more adaptor sequences, which can also be referred to as a universal sequence or a common sequence. The adaptor sequence can enable barcoding of the amplicon in a subsequent reaction. Alternatively, or in addition, the PCR primers, or a subset of the PCR primers, used in target specific PCR can include one or more barcodes. For example, the primers can include a sample barcode (e.g., a barcode that is common to all primers used on a particular sample, reaction, or amplicon), a sequence barcode (e.g., a unique barcode), a universal sequencing primer binding sequence, or a combination thereof. A sample barcode can also be referred to as a multiplex identifier (MID).

In some cases, such as when the yield of nucleic acids may be insufficient for further processing, a non-specific amplification reaction (e.g., non-specific PCR) can be performed on the Nucleic Acid Sample 820 to produce an Amplified Nucleic Acid Sample 830. As illustrated in FIG. 8, this reaction can occur prior to target specific amplification to produce the Amplified (VH)4 Sequences 850. The method used for a non-specific amplification can depend upon the type of nucleic acids in the Nucleic Acid Sample. For example, whole genome amplification (WGA) can be performed on genomic DNA. The non-specific amplification can use a pool of random primers (e.g., random hexamer primers).

In some cases, an Amplified Nucleic Acid Sample can be subjected to one or more cleaning steps to produce a Clean Amplified Nucleic Acid Sample 840, as shown in FIG. 8. For example, primers or other components of the non-specific PCR reaction can be removed. Any method can be used to clean the Amplified Nucleic Acid Sample 830. For example, gel purification can be performed. In another example, a commercial kit for cleaning a PCR reaction (e.g., QiaAmp Micro, Agencourt AMPure XP).

The Amplified (VH)4 Sequences 850 can be sequenced using any suitable method. The sequencing method can be a next generation sequencing method. The next generation sequencing method can be, for example, Helicos True Single Molecule Sequencing (tSMS), 454 sequencing, SOLiD technology sequencing, SOLEXA sequencing, single molecule, real-time (SMRT™) sequencing, nanopore sequencing, semiconductor sequencing, DNA nanoball sequencing, or any other next generation sequencing technique. In some embodiments, 454 sequencing is used to sequence the Amplified (VH)4 Sequences.

In some cases, as illustrated in FIG. 8, one or more barcodes can be added to the Amplified (VH)4 Sequences 850 prior to sequencing (e.g., next generation sequencing) to produce Barcoded (VH)4 Sequences 860. For example, a sample barcode (e.g., a barcode that is common to all primers used on a particular sample, reaction, or amplicon), a sequence barcode (e.g., a unique barcode), a universal sequencing primer binding sequence, or a combination thereof can be added. A sample barcode can also be referred to as a multiplex identifier (MID). The use of a sample barcode (e.g., a MID) can enable multiple samples to be combined in a single sequencing run. The sequence barcode can enable multiple sequencing reads of the same nucleotide to be identified and aligned. The one or more barcodes can be on a single fusion primer. The fusion primer can also contain a sequence that recognizes adaptor sequence (which can also be referred to as a universal sequence or a common sequence) that was included in the target specific PCR primers.

As illustrated in FIG. 8, the Barcoded (VH)4 Sequences 860 can be subjected to one or more cleaning steps to produce Clean Barcoded (VH)4 Sequences 870 prior to sequencing. For example, primers or other components of the barcode PCR reaction can be removed. Any method can be used to clean the Sample Barcoded (VH)4 Sequences 860. For example, gel purification can be performed. In another example, a commercial kit for cleaning a PCR reaction (e.g., QiaAmp Micro, Agencourt AMPure XP) can be used.

As illustrated in FIG. 8, the amount of nucleic acids in a Barcoded (VH)4 Sequences 860 or Clean Barcoded (VH)4 Sequences 870 can be normalized prior to combining with nucleic acids from other samples to produce a Normalized Sample Pool 880 prior to sequencing. The nucleic acids can be quantitated prior to normalization using any method (e.g., spectrometry, or using a kit).

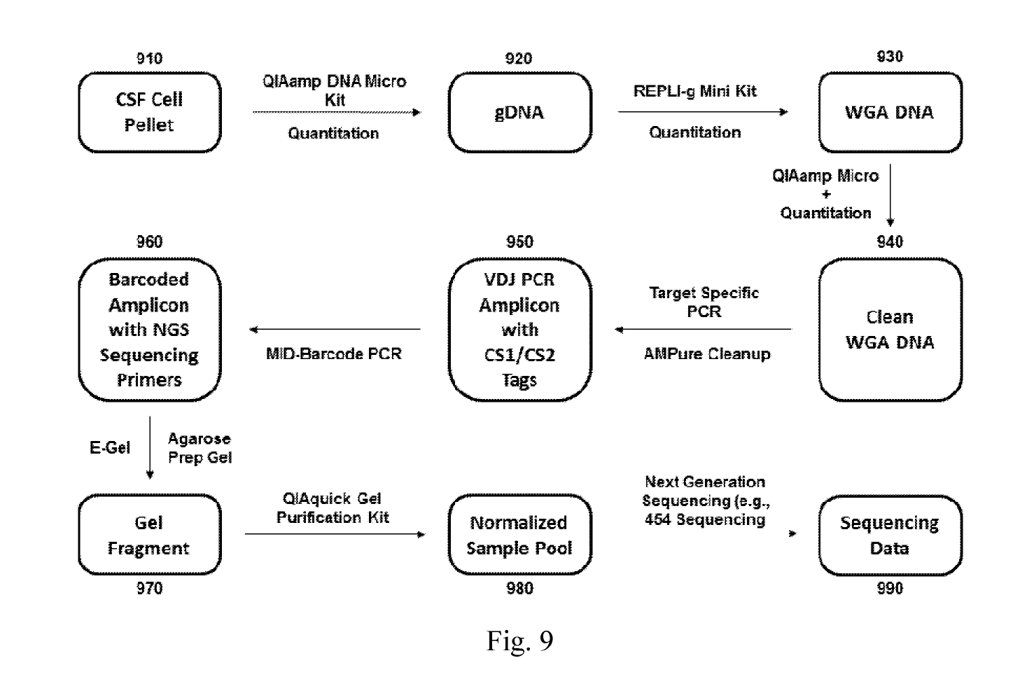

FIGS. 9-13 illustrate exemplary protocols for processing a CSF Cell Pellet 910 to produce Sequencing Data 990 using next generation sequencing (e.g., 454 Sequencing). Specific numbers or amounts indicated in the figures and the accompanying description are exemplary amounts, and can be scaled or changed as needed.

As illustrated in FIG. 9, genomic DNA (gDNA) 920 can be isolated from a CSF Cell Pellet 910 from a subject sample. The gDNA 920 can be isolated, for example, using a QIAamp DNA Micro Kit. The amount of gDNA 920 isolated can be quantitated.

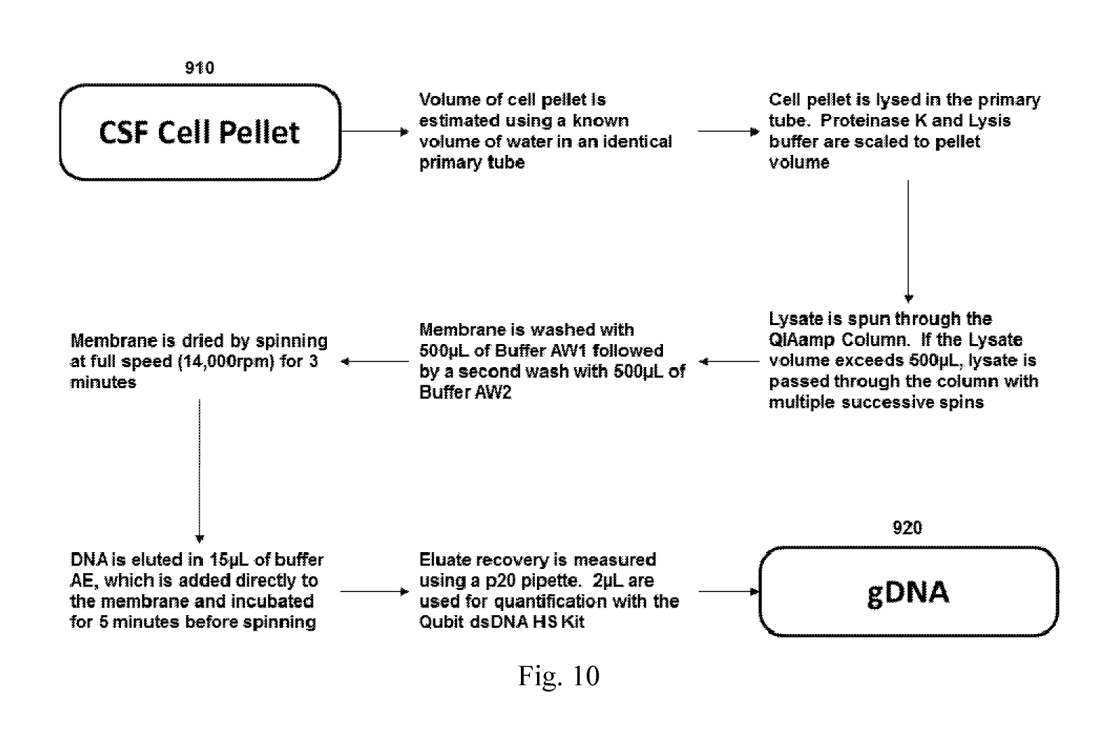

A more detailed exemplary process for isolating gDNA 920 from a CSF Cell Pellet 910 is illustrated in FIG. 10. The volume of the CSF Cell Pellet 910 can be estimated using any number of techniques. In this exemplary process, the volume of the CSF Cell Pellet 910 is estimated using a known volume of water in an identical primary tube. The cell pellet volume can also be estimated by using a liquid other than water, such as a suitable buffer. Once the volume is determined, the cell pellet is lysed. In this exemplary process, the lysis is performed using proteinase K in a lysis buffer. Such reagents can be scaled with regard to the pellet volume. Lysis can also be performed using other techniques known in the art. The cell lysate can be centrifuged through a column. A column can be any suitable column. In the exemplary process of FIG. 10, a QIAamp column is used. In some cases, lysate volume can exceed a column's capacity. In such cases, a lysate can be passed through the column multiple times. A column's maximum capacity can be 500 microliters for example.

Upon completion of a centrifugation step comprising cell lysate, a column's membrane can be washed. A column membrane can be washed using a variety of reagents. For example, a membrane can be washed with AW1 buffer and/or AW2 buffer. In some cases, successive washes can be performed with AW1 buffer and AW2 buffer. Any volume of buffer can be used in a wash step. A suitable volume can be 500 μL. A suitable volume can be over 500 μL. In some cases, a volume of buffer can be adjusted based on the quality or amount of cell lysate being washed. Once a column membrane has been washed, a membrane can be dried. A membrane can be dried by centrifugation or by allowing the membrane to dry at room temperature. If centrifugation is being used for drying of a membrane, any suitable speed and amount of time can be used. For example, a membrane can be dried by centrifugation at 14,000 rpm for 3 minutes. A membrane can be dried at less than 14,000 rpm or greater than 14,000 rpm. A membrane can also be dried for over 3 minutes or less than 3 minutes.

A dried column membrane can be further processed with any number of steps. For example, DNA can be eluted from a dried membrane in buffer or water. A suitable buffer can be AE buffer. A buffer can be added directly to a membrane and incubated. A suitable buffer can also be added to a membrane and not incubated. DNA can be eluted from a membrane with AE buffer and centrifuged for any amount of time. DNA can be eluted from a membrane with AE buffer and centrifuged for approximately 5 minutes or exactly 5 minutes. Upon DNA recovery from a column membrane, an eluate recovery can be measured. Measuring can be performed using a pipette. Measuring can also be performed using estimation. In some cases, measuring eluate recover can be done with a Qubit dsDNA HS kit. Eluate can comprise genomic DNA. Eluate can comprise other nucleic acids.

Returning to FIG. 9, gDNA 920 can be subjected to whole genome amplification (WGA) to produce WGA DNA 930. WGA can be performed on small samples or samples with low DNA concentration. WGA can also be performed on samples with poor DNA quality. WGA can be performed on any type of sample containing genomic DNA. WGA can be performed using multiple displacement amplification (MDA). WGA can be a non-PCR based DNA amplification technique. In some cases, WGA can rapidly amplify minute or poor amounts of DNA to an acceptable quantity for genomic analysis. The WGA can be performed using a REPLI-g Mini Kit. The WSG DNA 930 can be quantitated. The WGA DNA 930 can be cleaned (e.g., using a QIAamp Micro Kit, gel fragment purification, etc.) to produce Clean WGA DNA 940. The Clean WGA DNA 940 can be quantitated.

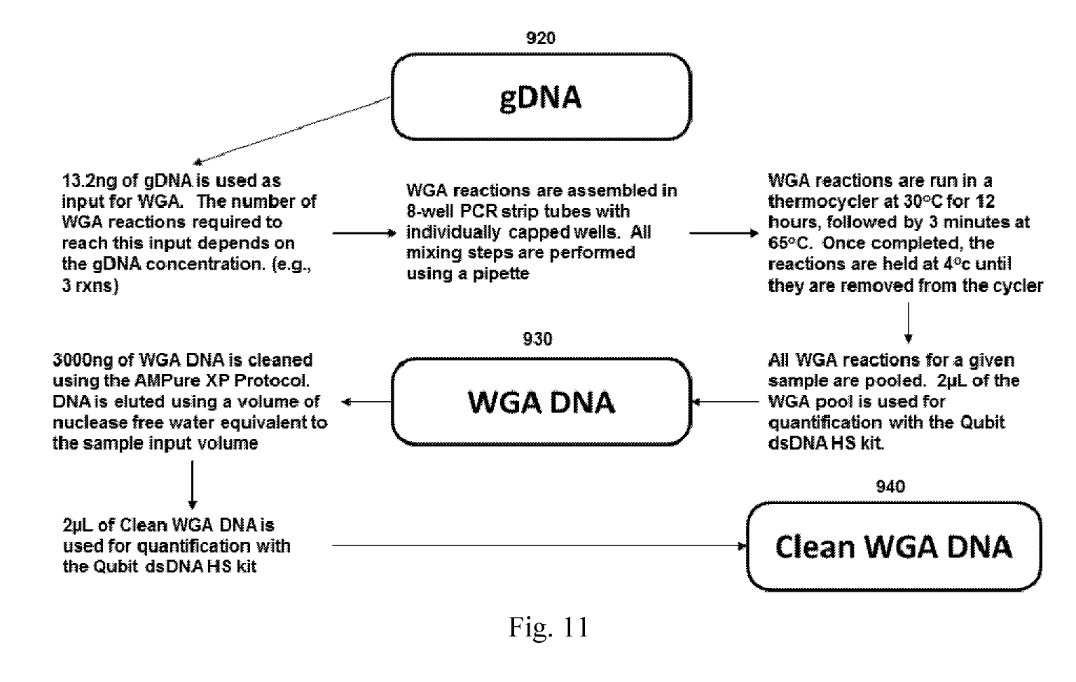

A more detailed exemplary process for generating and cleaning WGA DNA is illustrated in FIG. 11. As shown in FIG. 11, an amount of gDNA (e.g., 13.2 ng) can be used as an input for WGA. Depending upon the concentration of the gDNA 920, multiple WGA reaction may be used (e.g., 3 reactions can be used). In some cases, 3 or fewer WGA reactions are used. In other cases, over 3 reactions may be needed to reach a suitable input. WGA reactions can be assembled in PCR strip tubes. PCR strip tubes can contain any number of wells. 8-well PCR strip tubes can be used, for example. PCR strip tubes can be individually capped or not. PCR strip tubes with loaded reagents can be mixed using a variety of techniques. In some cases, a pipette can be used for mixing. In other cases, a strip tube can be mixed with a vortex followed by centrifugation.

As shown in FIG. 11, WGA reactions can be performed in a thermocycler to produce WGA DNA 930. Any suitable PCR program can be used. For example, as illustrated in FIG. 11, a thermocycler can be programmed at 30° C. for 12 hours, followed by 3 minutes at 65° C. Upon completion of the protocol, reactions can be held at 4° C. until they are removed from the cycler. WGA reactions for an individual sample can be pooled. In some cases, 2 microliters of a WGA pool is used for quantification. Quantification can be performed with a Qubit dsDNA HS kit.

As shown in FIG. 11, WGA DNA 930 can be cleaned using one or more cleaning steps to remove, for example, unused primers or other components of the WGA reaction. An amount of WGA DNA 930 (e.g., 3000 ng) of WGA DNA can be cleaned using an AMPure XP protocol. Any other suitable protocol can replace the AMPure XP protocol. DNA can be eluted using any volume of nuclease free water equivalent to a sample input volume. DNA can also be eluted using buffer. Clean WGA DNA can further be quantified. Quantification of WGA DNA can be performed with a Qubit dsDNA HS kit or any suitable kit.

Returning to FIG. 9, target specific PCR can be performed on Clean WGA DNA 940 to produce VDJ PCR Amplicon with CS1/CS2 Tags 950. Target specific PCR primers can be used to amplify immunoglobulin heavy chain variable (VH)-diversity (DH)-joining (JH) (VDJ) regions. As illustrated in FIG. 9, VDJ PCR amplicons can be tagged. A tag can be a common sequence 1 and/or a common sequence 2, CS1 and CS2, respectively. VDJ PCR amplicons can be amplified with any type of tag. The target specific PCR can be performed using degenerate target-specific PCR primers, non-degenerate target-specific PCR primers, or a combination thereof. The target specific PCR can be a two-step nested PCR reaction, or a single step PCR reaction. In some embodiments, a two-step nested PCR protocol and degenerate target-specific PCR primers is used. In some embodiments, a two-step nested PCR protocol using pools of non-degenerate PCR target-specific PCR primers is used. In some embodiments, a single PCR reaction incorporating a pool of non-degenerate target-specific PCR primers is used. The PCR primers can be designed based upon an alignment of known (VH)4 sequences. The PCR primers, or a subset of the PCR primers, used in target specific PCR can include one or more adaptor sequences, which can also be referred to as a universal sequence or a common sequence. The adaptor sequence can enable barcoding of the amplicon in a subsequent reaction. Alternatively, or in addition, the PCR primers, or a subset of the PCR primers, used in target specific PCR can include one or more barcodes. For example, the primers can include a sample barcode (e.g., a barcode that is common to all primers used on a particular sample, reaction, or amplicon), a sequence barcode (e.g., a unique barcode), a universal sequencing primer binding sequence, or a combination thereof. A sample barcode can also be referred to as a multiplex identifier (MID).

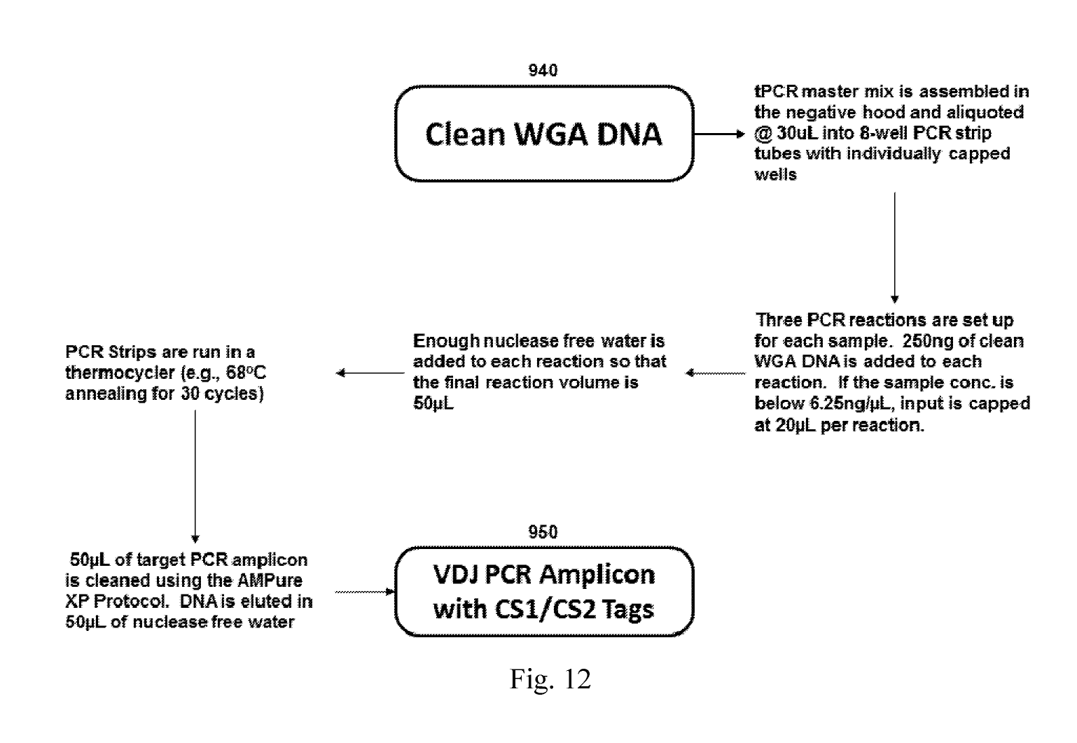

A more detailed exemplary process for producing VDJ PCR Amplicon with CS1/CS2 Tags 950 from Clean WGA DNA 940 is shown in FIG. 12. As shown in FIG. 12, a tPCR master mix can be prepared in a negative hood and aliquoted into PCR strip tubes. The tPCR master mix can be of any reaction volume (e.g., 30 μL). One or more reactions can be set up (e.g., 3 reactions) for each Clean WGA DNA 940 sample. An amount of Clean WGA DNA 940 can be added to each reaction. For example, about 250 ng of Clean WGA DNA 940 can be added to each reaction. In some cases, when the concentration of the Clean WGA DNA is low, the volume added to an individual reaction may need to be capped. For example, the volume of Clean WGA DNA that is added to 30 μL of tPCR master mix can be capped at 20 μL if the concentration of the Clean WGA DNA is below 6.25 ng/μL. Nuclease free water can be subsequently added to each reaction to reach a constant final volume; for example, to bring the total reaction volume to be about 50 μL. The PCR Strips can be loaded in a thermocycler and amplified using a suitable protocol (e.g., 68° C. annealing temperature with 30 cycles of extension) to produce VDJ PCR Amplicon with CS1/CS2 Tags. 950 Upon completion of a thermocycler protocol, target PCR amplicons can be cleaned using any method. For example, a suitable method can entail using an AMPure XP kit. DNA can be eluted in water or buffer at any volume. A suitable volume can be about 50 μL.

Returning to FIG. 9, one or more barcodes can be added to the VDJ PCR Amplicon with CS1/CS2 Tags 950 in a MID-Barcode PCR to produce Barcoded Amplicon with NGS Sequencing Pimers 960. For example, a sample barcode (e.g., a barcode that is common to all primers used on a particular sample, reaction, or amplicon), a sequence barcode (e.g., a unique barcode), a universal sequencing primer binding sequence (e.g., NGS Sequencing Primers), or a combination thereof can be added. A sample barcode can also be referred to as a multiplex identifier (MID). The use of a sample barcode (e.g., a MID) can enable multiple samples to be combined in a single sequencing run. The sequence barcode can enable multiple sequencing reads of the same nucleotide to be identified and aligned. The one or more barcodes can be on a single fusion primer. The fusion primer can also contain a sequence that recognizes adaptor sequence (which can also be referred to as a universal sequence or a common sequence) that was included in the target specific PCR primers.

As illustrated in FIG. 9, Barcoded Amplicon with NGS Sequencing Pimers 960 can be electrophoresed on an agarose gel to separate the amplified sequences from primer sequences. Gel Fragments 970 containing bands of the appropriate size can be excised from the gel. DNA can be extracted from the Gel Fragment 970 using any known method. For example, a QIAquick Gel Purification Kit can be used.

As illustrated in FIG. 9, samples from multiple subjects can be normalized and combined in a Normalized Sample Pool 980 and then sequenced using Next Generation Sequencing (NGS) (e.g., by 454 Sequencing) to generate Sequencing Data 990.

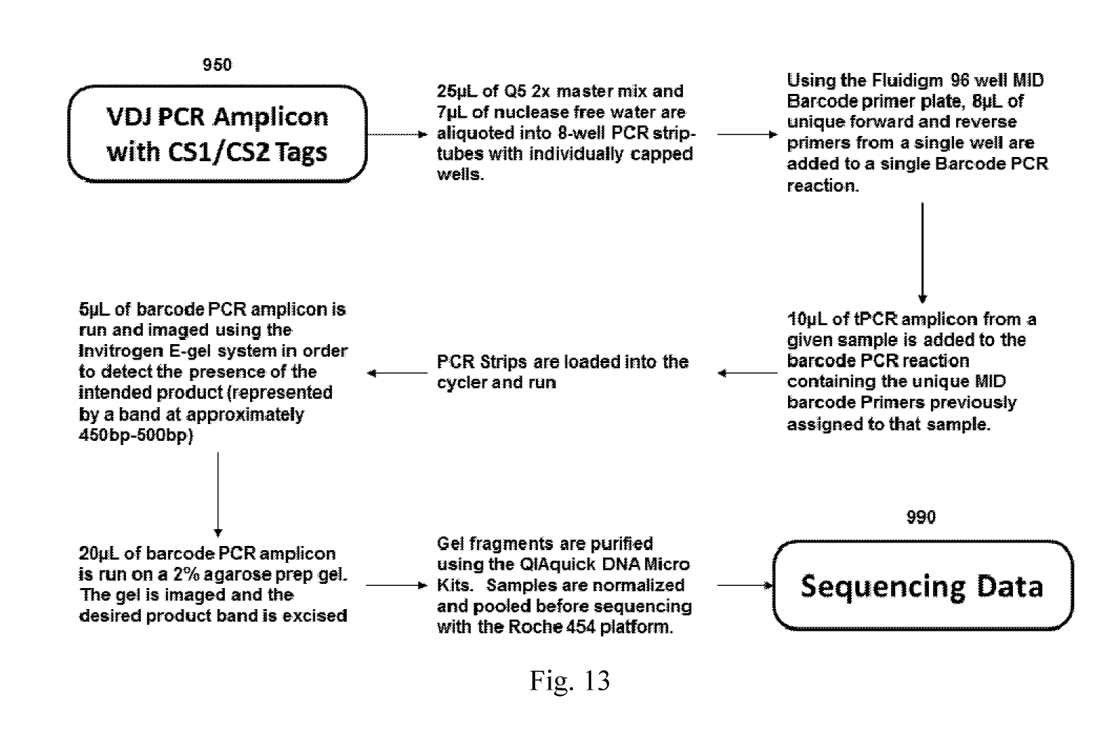

More details of an exemplary protocol for generating Sequencing Data 990 from VDJ PCT Amplicon with CS1/CS2 Tags 950 are illustrated in FIG. 13. Barcode PCR reactions are set up with an amount of a Q5 2× master mix (e.g., 25 μL) and an amount of nuclease free water (e.g., 7 μL). The reactions can be set up in individual PCR tubes or in strips of PCR tubes. The barcode PCR reactions can utilize a Fluidigm 96 well MID barcode primer plate. An amount (e.g., 8 μL) of unique forward and reverse primers from a single well of the Fluidigm 96 well MID barcode primer plate is added to a single barcode PCR reaction. An amount (e.g., 10 μL), 10 microliters of tPCR amplicon (e.g., VDJ PCR Amplicon with CS1/CS2 Tags) from a given sample can be added to the barcode PCR reaction. The barcode PCR reaction(s) can be run on a thermocycler using any suitable cycling parameter.

Barcoded amplicons with next generation sequencing primers can be cleaned by running a portion of the completed barcoding PCR reaction on an agarose gel and excising a gel fragment containing an appropriately sized band. As illustrated in FIG. 13, a test gel is run using a small amount (e.g., 5 μL) of barcode PCR amplicon is run and imaged using the Invitrogen E-gel system in order to detect the presence of the intended product (represented by a band at approximately 450 bp-500 bp). Then, a larger amount (e.g., 20 μL) of barcode PCR amplicon is run on a prep gel (e.g., a 2% agarose prep gel). The gel is imaged and the desired product band is excised. Gel fragments can be frozen and shipped to a centralized sequencing provider. Alternatively, gel fragments can be processed on site. Gel fragments can be purified using a QIAquick DNA Micro Kit, or any other suitable method. Multiple samples can then be normalized and pooled before sequencing (e.g., with the Roche 454 platform) to produce Sequencing Data 990.

Processing Sequence Reads

The methods disclosed herein can comprise processing a set of sequence reads to generate a set of (VH)4 sequences.

Raw sequence reads from next generation sequencing can be processed and analyzed using computer analysis tools (e.g., the VDJserver online repertoire analysis tool). The processing and analysis can be as described previously in this specification, and can include trimming sequences, aligning sequence to each other, aligning sequences to know germline gene sequences, removing or filtering sequences, or a combination thereof.

(VH)4 antibody gene specific criteria can also be used to remove or filter sequences. For example, sequences missing a CDR3 sequence can be removed. In another example, sequences that are missing read coverage between Chothia-numbered codons 31-92 can be removed. Additionally, sequences that do not align to a VH4 sequence can be removed.

CDR3 segment matching from sample to sample should not occur. Highly amplified sequences present in multiple samples (identified by their matching CDR3 nucleotide segment) can therefore be removed. However, sequences containing matching CDR3 sequences can be retained where the majority of that sequence (e.g., >99%) is represented in a single sample.

Following processing of the raw sequence reads, a set of (VH)4 sequences is produced.

Analyzing (VH)4 Sequences

Mutation Analysis and Composite Signature Score

Unique VH4 sequences can be analyzed to determine a mutation frequency at one or more codons. For example, mutation analysis can be performed on one or more codons in the region between codons 31 and 92 following the Chothia numbering system. The mutation analysis can use the framework and complementarity determining regions originally defined by Kabat.

Mutation analyses can be performed at the nucleotide level, the codon level, or both. Mutations in a codon that result in an amino acid substitution are referred to as replacement mutations (RM).

The replacement mutation frequency (RMF) at two or more codons can be used to calculate an antibody gene signature (AGS) (also referred to herein as a Composite signature score). AGS scores are the sum for each AGS codon (31b; 40; 56; 57; 81) of [RMF at the AGS codon minus the average RMF (1.6) in a healthy control peripheral blood database divided by the standard deviation (0.9) of the average RMF of the same healthy control database].

An ACS or composite signature score can be calculated based upon the RMF at two or more codon positions. The two or more codon positions can be selected from codons 24 to 95, or from codons 31 to 92. In some embodiments, the two or more codon positions can be selected from the group consisting of 31B, 40, 56, 57, 81, and 89. In some embodiments, the two or more codons can be selected from the group consisting of 31B, 40, 56, 57, and 81. In some embodiments, the two or more codons can include 31B, 40, 56, 57, and 81.

A composite signature score above a certain threshold can support a diagnosis of MS in a subject. For example, a composite signature score that is greater than about: 0.8, 1.8, 2.8, 3.8, 4.8, 5.8, 6.8, 7.8, 8.8, 9.8, 10.8, 11.8, or 12.8 can support a diagnosis of MS in a subject. In some embodiments, a composite signature score that is greater than about 6.8 supports a diagnosis of MS in a subject. In some embodiments, a composite signature score that is greater than about 7.8 supports a diagnosis of MS in a subject.

A composite signature score below a certain threshold can support a diagnosis of not having MS in a subject. For example, a composite signature score that is less than about: 0.8, 1.8, 2.8, 3.8, 4.8, 5.8, 6.8, 7.8, 8.8, 9.8, 10.8, 11.8, or 12.8 can support a diagnosis of not having MS in a subject. In some embodiments, a composite signature score that is less than about 4.8 supports a diagnosis of not having MS in a subject. In some embodiments, a composite signature score that is greater than about 5.8 supports a diagnosis of not having MS in a subject.

A composite signature score can yield an indeterminate result. For example, a composite signature score that is about: 0.8-10.8, 1.8-9.8, 2.8-8.8, 3.8-7.8, 4.8-6.8, 0.8-12.8, 1.8-11.8, 2.8-10.8, 3.8-9.8, 4.8-8.8, or 5.8-7.8 can be an indeterminate result. In some embodiments, a composite signature score that is about 4.8-6.8 is an indeterminate result. In some embodiments, a composite signature score that is about 5.8-7.8 is an indeterminate result. An indeterminate result can indicate that the subject should be monitored closely (e.g., with subsequent testing). An indeterminate result can indicate that the subject should not currently be treated for MS.

Selecting Samples as Generating Reportable Results

The methods disclosed herein can include the step of selecting the subject sample as suitable for diagnostic testing, reporting, or diagnostic testing and reporting when one or more of the following sample quality indicators are met: (i) the set of (VH)4 sequences are from more than a first threshold number of (VH)4 genes, (ii) the set of (VH)4 sequences are from a second threshold number to the first threshold number of (VH)4 antibody genes, and a diversity index for the set of (VH)4 sequences is greater than a diversity index threshold, wherein the second threshold number is less than the first threshold number, (iii) greater than or equal to a first threshold percentage of the set of sequence reads are (VH)4 sequences, (iv) less than or equal to a second threshold percentage of the set of sequence reads contain a CDR3 sequence identical to another sample, or (v) a composite signature score for the set of (VH)4 sequences is not an indeterminate result. In some embodiments, the subject sample is selected when two or more of the sample quality indicators are met. In some embodiments, the subject sample is selected when three or more of the sample quality indicators are met. In some embodiments, the subject sample is selected when four of the sample quality indicators are met.

In order to ensure that a sample contained a sufficient amount of B cells, or to ensure that nucleic acids isolated from the sample were not lost during processing, a minimum number of (VH)4 gene sequences can be required for generating a reportable result. For example, if a sample contains more than a first threshold number of (VH)4 gene sequences, that can be an indication that the sample is suitable for generating a reportable result. The first threshold can be, for example, about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the first threshold number of (VH)4 genes is about 30. In some embodiments, the first threshold number of (VH)4 genes is about: 10-50, 20-40, or 25-35. In some embodiments, the first threshold number of (VH)4 genes is about: 25-35.

Sometimes a sample may not contain a number of sequences to indicate, on its own, that the sample is suitable for generating a reportable result. In such cases, a diversity index can be calculated for that sample, and used to determine whether the sample is suitable.

As used herein the term “diversity index” refers to a VH4 diversity calculation based on the Shannon Wiener diversity index. Rather than simply counting the number of VH4 sub families present in a given sample as a means to quantify diversity, the diversity index provides a representation for the evenness of the distribution of all the individual VH4 genes identified in a sample across all known VH4 subfamilies. The index is calculated as follows:

$${H}^{′} = - {\sum }_{i = 1}^{R} {p}_{i} ln {p}_{i}$$

Wherein pᵢ is the proportion of the total number of VH4 genes identified represented by a given subfamily, and R is the total number of species in the subfamily.

An example of a diversity index calculation is provided below in Table 1.

For example, a sample can be suitable for reporting when the set of (VH)4 sequences are from a second threshold number to the first threshold number of (VH)4 antibody genes, and a diversity index for the set of (VH)4 sequences is greater than a diversity index threshold, wherein the second threshold number is less than the first threshold number.

The second threshold number of (VH)4 antibody genes can be about: 1, 2, 3, 4, 5, 6, 7, 8, or 9. The second threshold number of (VH)4 antibody genes can be about 5. The second threshold number of (VH)4 antibody genes can be about: 1-9, 2-8, 3-7, or 4-6. The second threshold number of (VH)4 antibody genes can be about 4-6.

The diversity index threshold can be about: 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0. In some embodiments, the diversity index threshold is about: 1.0. The diversity index threshold can be about: 1.0-5.0, 1.0-4.0, 1.0-3.0, 1.0-2.5, 1.0-2.0, or 1.0-1.5. In some embodiments, the diversity index threshold is about 0.85-1.15.

Another criteria that can be used in determining whether a subject sample can be selected as suitable for diagnostic testing, reporting, or diagnostic testing and reporting is the percentage of the set of sequence reads that are (VH)4 sequences. For example, the subject sample can be selected when greater than or equal to a first threshold percentage of the set of sequence reads are (VH)4 sequences. The first threshold percentage can be about: 5%, 10%, 15%, 20%, 25%, 30%, 35%, 40%, 45%, 50%, 55%, 60%, 65%, 70%, 75%, 80%, 85%, 90%, 95% or 99%. In some embodiments, the first threshold percentage is about 55%. In some embodiments, the first threshold percentage is about 60%. The first threshold percentage can be about: 5-99%, 10-95%, 15-90%, 20-85%, 25-80%, 30-75%, 35-70%, 40-65%, 45-60%, or 50-60%. In some embodiments, the first threshold percentage is about 40-65%. In some embodiments, the first threshold percentage is about 50-60%.

Because CDR3 segment matching from sample to sample should not occur, another criteria that can be used in determining whether a subject sample can be selected as suitable for diagnostic testing, reporting, or diagnostic testing and reporting is the percentage of sequence reads in the sample that contain a CDR3 sequence that is identical to another sample. Too high of a percentage can indicate that cross contamination between samples has occurred. For example, the subject sample can be selected as suitable for diagnostic testing, reporting, or diagnostic testing and reporting when less than or equal to a second threshold percentage of the set of sequence reads contain a CDR3 sequence identical to another sample. The second threshold percentage can be about: 5%, 10%, 15%, 20%, 25%, 30%, 35%, 40%, 45%, 50%, 55%, 60%, 65%, 70%, 75%, 80%, 85%, 90%, 95% or 99%. In some embodiments, the second threshold percentage is about 50%. The second threshold percentage can be about: 5-99%, 5-95%, 10-90%, 15-85%, 20-80%, 25-75%, 30-70%, 40-60%, or 45-55%. In some embodiments, the second threshold percentage is about 40-60%. In some embodiments, the second threshold percentage is about 45-55%.

Another criteria that can be used in determining whether a subject sample can be selected as suitable for diagnostic testing, reporting, or diagnostic testing and reporting is whether or not a codon signature score for that sample is considered an indeterminate result. For example, the subject sample can be selected as suitable for diagnostic testing, reporting, or diagnostic testing and reporting when the composite signature score is not an indeterminate result. An indeterminate result can be a composite signature score that is about: 0.8-10.8, 1.8-9.8, 2.8-8.8, 3.8-7.8, 4.8-6.8, 0.8-12.8, 1.8-11.8, 2.8-10.8, 3.8-9.8, 4.8-8.8, or 5.8-7.8 can be an indeterminate result. In some embodiments, a composite signature score that is about 4.8-6.8 is an indeterminate result. In some embodiments, a composite signature score that is about 5.8-7.8 is an indeterminate result. An indeterminate result can indicate that the subject should be monitored closely (e.g., with subsequent testing). An indeterminate result can indicate that the subject should not currently be treated for MS.

In another aspect, the present disclosure provides a method for identifying a subject that has or is likely to develop MS. In one embodiment, the method comprises determining a composite signature score and/or a diversity index score based on the nucleotide sequences in the variable heavy chain (VH4) antibody genes from a sample collected from the subject. In one embodiment, the method comprises obtaining a sample from the subject, isolating DNA or RNA from the sample, determining nucleotide sequences in the sample for a set of VH4 antibody genes (e.g., selected from genes at codons 24 to 95), and identifying mutations with respect to germ-line VH4 sequences, thereby determining amino acid replacement frequency at codon positions across the VH4 region. In a further embodiment, the method comprises calculating a composite signature score, wherein the composite signature score comprises the sum of replacement frequencies at a plurality of codon positions; and calculating the VH4 gene diversity index, wherein the VH4 gene diversity index is calculated by determining the sum of the diversity of distribution among all of the individual VH4 genes identified in the sample. In a further embodiment, the method comprises comparing the composite signature score to a pre-determined threshold value. In a yet further embodiment, the method comprises diagnosing the subject as having MS or as likely to develop MS if the composite score exceeds the threshold value or if the composite score falls below the threshold value, but the diversity index score is greater than 1. In one embodiment, the method comprises diagnosing the subject as having MS or as likely to develop MS if the composite score exceeds the threshold value or if the composite score falls below the threshold value, but the diversity index score is from about 0.75 to about 5, from about 1 to about 5, from about 1 to about 4, from about 1 to about 3, from about 1 to about 2, from about 1 to about 1.5, from about 1 to about 1.2, or from about 1 to about 1.1.

In one embodiment, the composite signature is calculated from the replacement frequencies at codons 31B, 40, 56, and 57. In a further embodiment, the composite signature is calculated from the replacement frequencies at codons 31B, 40, 56, 57, and 81. In a yet further embodiment, the composite signature is calculated from the replacement frequencies at codons 31B, 40, 56, 57, 81, and 89.

In one embodiment, the amino acid replacement frequency at each codon position is normalized to the average replacement frequency of the corresponding region of VH4 antibody genes in healthy controls prior to calculation of the composite signature score. In another embodiment, the predetermined threshold value is determined from the average replacement frequencies of a plurality of codons in VH4 antibody genes from CSF samples of a cohort of multiple sclerosis patients.

In one embodiment, the sample is a cerebrospinal fluid (CSF) sample. In another embodiment, the sample is a peripheral blood sample.

In one embodiment, the subject presents with symptoms of a neurological disorder or demyelinating disease. In another embodiment, the subject presents with Clinically Isolated Syndrome. In another embodiment, the subject has experienced a demyelinating event. In one embodiment, the subject is positive for oligoclonal bands. In another embodiment, the subject is negative for oligoclonal bands. In one embodiment, the subject is receiving treatment with one or more immunomodulatory drugs. In a further embodiment, the immunomodulatory drug can be a steroid, Avonex, Betaseron, Rebif, Campath, Copaxone, Cellcept, Tacrolimus, or Rapamune.

In one embodiment, the method further comprises identifying the subject to receive a therapeutic regimen for treating MS based on the composite signature score and/or the diversity index. In a further embodiment, the therapeutic regimen is a B cell depletion therapy or interferon therapy.

Computer Implementation

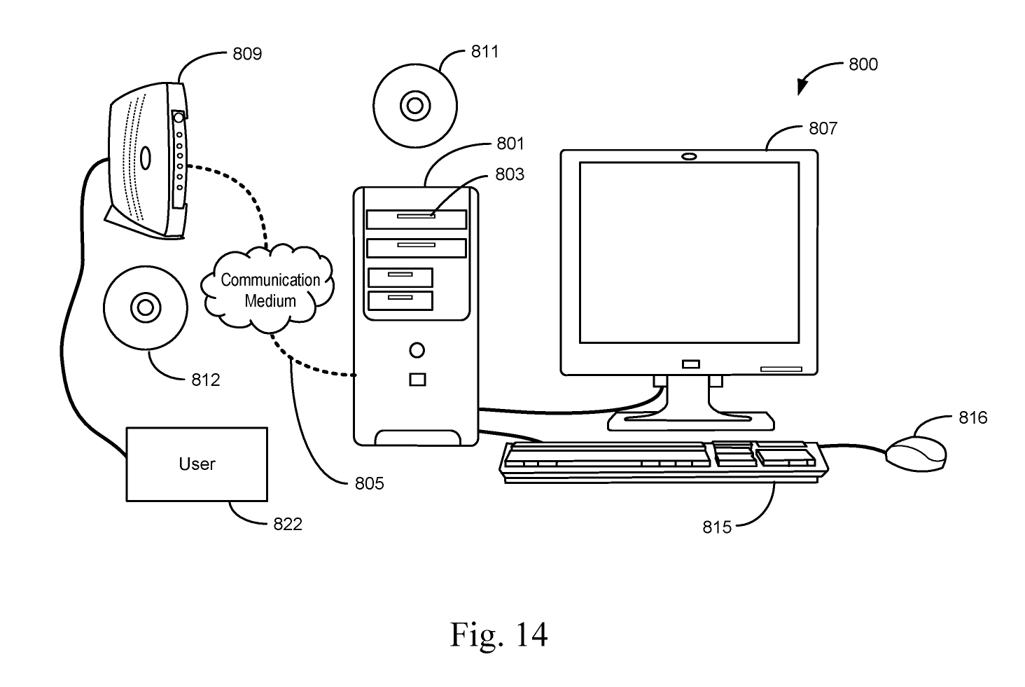

One or more computers may be utilized in the methods disclosed herein, such as a computer 800 as illustrated in FIG. 14. The computer 800 may be used for managing subject and sample information such as sample or subject tracking, database management, analyzing sequencing data, analyzing cytological data or other data provided by a physician, storing data, billing, marketing, reporting results, or storing results. The computer may include a monitor 807 or other graphical interface for displaying data, results, billing information, marketing information (e.g. demographics), subject information, or sample information. The computer may also include means for data or information input 816, 815. The computer may include a processing unit 801 and fixed 803 or removable 811 media or a combination thereof. The computer may be accessed by a user in physical proximity to the computer, for example via a keyboard and/or mouse, or by a user 822 that does not necessarily have access to the physical computer through a communication medium 805 such as a modem, an internet connection, a telephone connection, or a wired or wireless communication signal carrier wave. In some cases, the computer may be connected to a server 809 or other communication device for relaying information from a user to the computer or from the computer to a user. In some cases, the user may store data or information obtained from the computer through a communication medium 805 on media, such as removable media 812. It is envisioned that data or reports can be transmitted over such networks or connections for reception and/or review by a party. The receiving party can be, but is not limited to, an individual, a health care provider, or a health care manager. In one embodiment, a computer-readable medium includes a medium suitable for transmission of a result of an analysis of a biological sample, such as a (VH)4 Codon Signature. The medium can include a result regarding a diagnosis of having a taste or smell disorder for a subject, wherein such a result is derived using the methods described herein.

Sample information can be entered into a database for the purpose of one or more of the following: inventory tracking, assay result tracking, order tracking, subject management, subject service, billing, or sales. Sample information may include, but is not limited to: subject name, unique subject identification, subject-associated medical professional, indicated assay or assays, assay results, adequacy status, indicated adequacy tests, medical history of the subject, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a subject, medical professional, insurance provider, third party, or any individual or entity granted access. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or subject confidentiality.

### EXAMPLES

### Example 1

Studies were conducted to assess the presence of a mutation pattern in B cells collected from patients having MS in comparison to healthy controls or patients with neurological disorders other than MS. In initial studies, the control database used for comparison was a pool of memory B cells isolated from healthy donor peripheral blood (n=2). Additional comparisons to CSF B cell antibody repertoires from a limited number of patients with other neurological diseases (n=3) was also performed. Thus, further confirmation was required regarding the specificity of SHM accumulation at these codon positions in B cells from MS patients. In addition, the majority of patients analyzed in initial studies were patients who were very early in their disease. In fact, 17 out of 19 subjects included in two previous studies were classified as having clinically isolated syndromes (CIS) at the time their CSF was sampled. Thus, it was unclear whether MS patients who meet the McDonald criteria for MS and MS patients who are on or have recently been on steroid or other disease modifying therapies have the same pattern(s) of SHM at these codon positions.

To address these issues, we analyzed the VH4 antibody gene repertoires in CSF cell pellets from 26 patients with other neurological diseases (OND) and 13 patients with confirmed relapsing-remitting MS (RRMS) using next generation sequencing (NGS). The results indicate that RRMS patients and RRMS patients on disease modifying therapies (DMTs) exhibited the expected pattern of SHM at these codon positions. In addition, 23/26 OND patients did not appreciably accumulate SHM at these codon positions or displayed insufficient sequence data indicative of low B cell abundance in the CSF.

Materials and Methods

Patient Description and CSF Sample Preparation

CSF cell pellets were collected from 26 OND patients and 13 patients with confirmed or possible RRMS and the antibody repertoires were analyzed. All CSF samples were collected by lumbar puncture in accordance with IRB-approved protocols at UT Southwestern Medical Center, the University of Massachusetts Memorial Medical Center (UMass), John Hopkins University (JHU), or purchased from a commercial biorepository (PrecisionMed, Solana Beach, Calif.).

PCR and next generation sequencing of antibody genes from CSF-derived B cell pools

All PCR reactions and sequencing were performed as previously published (Rounds et al. Frontiers in Neurology 2014) with modifications made to account for usage of genomic DNA (gDNA).

Total CSF cell pellets were generated from 8-10 mL of CSF by centrifugation at 400× g and 4° C. The CSF supernatant was transferred to a fresh tube and frozen at −80° C. The cell pellet was resuspended in 400 μL RPMI cell culture medium, transferred to a 2 mL cryovial and centrifuged again. The cell-free supernatant was discarded and the CSF cell pellet was frozen at −80° C. until use.

Naive (CD19+CD27−) peripheral blood B cell pools were isolated from a healthy control sample and used in replicate as process controls to evaluate batch to batch variation and to aid in the evaluation of potential sequence errors generated during processing. Peripheral blood from a healthy control donor was collected in blood tubes containing heparin as an anti-coagulant (BD, Franklin Lakes, N.J.). Peripheral blood mononuclear cells (PBMCs) were isolated by centrifugation through Ficoll-Paque (GE Healthcare, PA). PBMCs were washed, counted and stained before being used to isolate naïve B cells as described previously (Ireland et al., JAMA 2014).

Amplification of IGHV4 sequences and next generation sequencing (NGS) was performed at SeqWright Genomic Services (a GE company, Houston, Tex.) starting with total CSF cell pellets. Genomic DNA (gDNA) was isolated using QIAamp DNA Micro Kits (Qiagen) and quantitated using the Quant-iT PicoGreen dsDNA Assay Kit (Invitrogen, Carlsbad, Calif.). Whole genome amplification (WGA) was performed using the REPLI-g Mini Kit (Qiagen) on up to 1000 cell equivalents of gDNA (6.6 ng) isolated from each clinical sample.

PCR amplification of IGHV4 sequences was performed using a modified nested PCR strategy using the 4-primer Amplicon Tagging strategy developed by Fluidigm (South San Francisco, Calif.) to allow for multiplex sequencing. All PCR reactions were performed using Phusion High-fidelity DNA Polymerase (New England Biolabs (NEB), Ipswich, Mass.) to minimize the generation of amplification errors.

Four external and four internal PCR reactions were performed for each sample to increase the total amount of subject DNA processed and minimize the chance of seeing any stochastic effects for CSF samples that have very low numbers of IGHV4-expressing B cells. Each external PCR reaction consisted of 125 ng of WGA DNA, 10.0 μL 2× Phusion DNA Polymerase Master mix (NEB), 1.0 μL, each of 10 μM pooled external forward and reverse PCR primers and water to bring the total volume to 20 μL. PCR cycling conditions were as follows: 98° C. for 3 minutes followed by 23 cycles of 98° C. for 10 seconds, 68° C. for 10 seconds, 72° C. for 10 seconds. The last 72° C. extension was extended to 10 minutes followed by a 4° C. hold. Each internal PCR reaction consisted of 3.0 μL DNA from the external PCR reaction, 10.0 μL 2× Phusion DNA Polymerase Master mix (NEB), 1.0 μL each of 10 μM pooled CS1/CS2-tagged internal forward and reverse PCR primers and water to bring the total volume to 20 μL. PCR cycling conditions were as follows: 98° C. for 1 minute followed by 10 cycles of 98° C. for 10 seconds, 68° C. for 10 seconds, 72° C. for 10 seconds then 21 cycles of 98° C. for 10 seconds, 72 ° C. for 10 seconds. The last 72° C. extension was extended to 10 minutes followed by a 4° C. hold.

An aliquot of each of the four internal PCR reactions for each subject was analyzed on a 2% agarose-TAE gel. Internal PCR reactions that yielded a visible band of the appropriate size were pooled for each subject, extracted, barcoded, quality controlled and sequenced as previously described (Owens et al. Neurological research 2006). Equimolar amounts of 454/barcode-tagged DNA from up to 21 clinical subjects plus controls were pooled and sequenced together in a single NGS run. A total of three sequencing runs were used to sequence all the samples for this study.

PCR was performed using Phusion High-fidelity DNA polymerase (New England Biolabs, Ipswich, Mass.) to minimize amplification errors. Next-generation DNA sequencing was performed at SeqWright Genomic Services, a GE company in Houston, Tex. using the 454 GS FLX DNA Sequencer and 454 Titanium chemistry (Roche/454, Branford, Conn.) according to the manufacturer's recommended protocols.

NGS 454 Data Processing

Each raw sequence was analyzed using the VDJserver online repertoire analysis tool (available online at https://vdjserver.org/). Unique reads were identified and filtered as follows.

Reads were trimmed of all primers and sample barcode sequences, and then aligned to each other within a sample allowing for a total edge mismatch of 5 nucleotides between sequences identified as matching. The number of copies of each unique sequence was kept as a sequence tag for subsequent analysis and filtering. Each unique sequence was aligned to germline gene segment sequences using the IgBlast aligner through VDJserver. Initial filtering removed any sequence which met at least one of the following criteria: mean sequence quality <35, length shorter than 200, frame-shifting insertions or deletions, out of frame junction, stop codon present, truncated read length, less than 85% homology to germline sequence, missing CDR3, missing read coverage between Chothia-numbered codons 31-92, not VH4 aligned. We also removed highly amplified sequences that were present in multiple samples (identified by their matching CDR3 nucleotide segment) because CDR3 segment matching from sample to sample should not occur. The exceptions were matching CDR3 containing sequences with >99% representation in a single sample, in which case we used this conservative cut-off to justify the sample source of the sequence, and only removed its CDR3 matches from the other sample databases. Lastly, we discarded unique sequence reads which had fewer than two copies in the raw sequence data. Samples were filtered prior to analysis: we required at least 10 unique reads after filtering for a sample to be included in our analysis and also required each sample to have at least >=55% of the raw reads be VH4 and >=50% of the raw reads not be removed due to matching CDR3s from another sample (these two filters were passed by all samples with at least 10 unique reads).

Samples with fewer than 10 unique reads remaining after filtering were not included in our primary analysis, but are shown in Tables 2 and 3. In total, we analyzed 17,231 unique CSF B cell-derived sequences from N=13 RRMS patients and N=26 OND patients and 13,633 healthy control naïve B cell-derived sequences from N=1 donor.

TABLE 2
RRMS full patient sample summary

| | | | | | | Time | | |
|---|---|---|---|---|---|---|---|---|
| Patient | | | Diagnosis | | AGS | with | | OCB |
| ID | Age^{a} | Gender | at tap | Diagnosis^{b} | Score | MS^{c} | Treatment | Status^{a} |
| MS01 | 35 | F | RRMS | RRMS | 11.2 | NR | | NR |
| MS02 | 27 | F | RRMS | RRMS | −3.54 | 1 | Steroids (1 | POS |
| | | | | | | | month) | |
| MS03 | 39 | F | RRMS | RRMS | 33.25 | 0 | None | NEG |
| MS04 | 26 | F | RRMS | RRMS | 11.14 | 76 | Capaxone | NR |
| | | | | | | | (5 years) | |
| MS05 | 31 | F | RRMS | RRMS | 6.56 | 25 | Avonex (9 | POS |
| | | | | | | | months) | |
| MS06 | 42 | F | RRMS | RRMS | 10.02 | 92 | CellCept (7 | NEG |
| | | | | | | | years) | |
| MS07 | 35 | M | RRMS | RRMS | 26.82 | 24 | Avonex (2 | NEG |
| | | | | | | | years) | |
| MS08 | 36 | F | Possible | RRMS | 5.37 | 0 | | NR |
| | | | MS | | | | | |
| MS09 | 31 | M | RRMS | RRMS | 18.5 | 0 | None | NR |
| MS10 | 23 | F | Possible | RRMS | −1.51 | 0 | | NR |
| | | | MS | | | | | |
| MS11 | 32 | F | RRMS | RRMS | 15.22 | 0 | None | POS |
| MS12 | 58 | F | Possible | RRMS | 7.46 | 0 | ?? | NR?? |
| | | | MS | | | | | |
| MS13* | 58 | M | Possible | RRMS | −8.89 | 0?? | Tysabri (4.5 | NR? |
| | | | MS | | | | years) | |

^{a}At time of sampling (yrs)
^{b}Most up-to-date available
^{c}At time of sampling (months)
^{d}If immunomodulatory and at time of sampling
*low unique sequence read count sample (<10)
Abbreviations:
NR, not reported;
OCB, oligoclonal bands;
RRMS, relapsing-remitting MS

TABLE 3
Non-RRMS full patient sample summary

| | | | | AGS |
|---|---|---|---|---|
| Patient ID | Age^{a} | Gender | Diagnosis^{b} | Score |
| OND01 | 37 | M | OND | 4.55 |
| OND02 | 61 | M | Dementia | −0.71 |
| OND03 | 54 | M | Stroke | 18.34 |
| OND04 | 65 | F | Dementia | 31.56 |
| OND05 | 52 | F | Headache | −4.12 |
| OND06 | 48 | F | Neurosarcoidosis | 4.74 |
| OND07 | NR | F | Headache | −8.89 |
| OND08 | 57 | F | PND | −7.66 |
| OND09 | 44 | F | Encephalitis | −1.67 |
| OND10 | 67 | F | PND | −2.55 |
| OND11 | 49 | F | Urge incontinence | 20.9 |
| OND12 | 52 | M | Alzheimer's | 5.04 |
| OND13 | 22 | F | Headache | 4.91 |
| OND14* | 25 | F | Headache | −8.89 |
| OND15* | 40 | F | Headache | −8.89 |
| OND16* | 72 | F | ALS | −8.89 |
| OND17* | 56 | M | CIDP | −8.89 |
| OND18* | 33 | F | Suspected Glioma, possible MS | −8.89 |
| OND19* | NR | F | Peripheral neuropathy, | −8.89 |
| | | | antiphospholipid syndrome | |
| OND20* | 23 | M | Headache, Chiari malformation | −8.89 |
| OND21* | 32 | F | Sensory Neuropathy | −8.89 |
| OND22* | 50 | F | Hodgkin's lymphoma | −8.89 |
| OND23* | 67 | F | PND | −8.89 |
| OND24* | 62 | F | Dementia | −8.89 |
| OND25* | 54 | F | Nondemyelinating optic neuropathy | −8.89 |
| OND26* | 58 | F | PND | −8.89 |
| HCN | NR | NR | NA | −0.65 |

^{a}At time of sampling (yrs)
^{b}Most up-to-date available
*low unique sequence read count sample (<10)
Abbreviations:
NR, not reported;
OND, other neurological disorder;
PND, paraneoplastic neurologic disorder;
ALS, amyotrophic lateral sclerosis;
CIDP, chronic inflammatory demyelinating polyneurophathy;
HCN, healthy control naive peripheral B cells.

Mutation Analyses

Mutation analyses were performed as previously published (Rounds et al. Frontiers in Neurology 2014). Unique VH4 sequences were analyzed in the region between codons 31 and 92 following the Chothia numbering system using the framework and complementarity determining regions originally defined by Kabat. Mutation analyses were performed both at the nucleotide level and codon level. Mutations in a codon that result in an amino acid substitution are referred to as replacement mutations (RM) and the replacement mutation frequency (RMF) at each codon is the basis for calculating antibody gene signature (AGS) scores as previously described except that only five codons are used here. AGS scores are the sum for each AGS codon (31b; 40; 56; 57; 81) of [RMF at the AGS codon minus the average RMF (1.6) in a healthy control peripheral blood database divided by the standard deviation (0.9) of the average RMF of the same healthy control database].

Statistical Analyses

Each sample was included in one of three cohorts (RRMS, OND or HCN) for statistical analyses using GraphPad Software 6.00 (San Diego, Calif., USA). VH4 and JH gene segment frequencies were compared by Chi-squared analysis using a representative pool of 100 sequences from each cohort (gene counts were set to the percent frequency values). Gene distributions were compared by Chi-squared analysis of the group of VH4 or JH gene frequencies and individual genes were compared by Chi-squared analysis of the sequences that were and were not aligned to that specific gene. AGS-contributing codon frequencies were individually compared by Chi-squared analysis. Mutation frequencies and AGS scores were compared across cohorts by Mann Whitney test (statistical significance for all methods was attributed to p-values <0.05). Interquartile range (IQR) for AGS scores was defined at the 25th and 75th percentile. Specificity, sensitivity and accuracy were calculated for AGS based on the follow-up diagnosis. Specificity was calculated as (# correct non RRMS assessments)/(# non RRMS samples); sensitivity was calculated as (# correct RRMS assessments)/(# RRMS samples); and accuracy was calculated as (# correct assessments)/(# samples).

Results

For this study, we generated VH4 antibody repertoires using next-generation DNA sequencing of CSF cell pellets isolated from 39 patients and a pool of purified CD19+CD27− naïve B cells from one healthy donor (run in 10 replicates) (Table 4). Of the 39 patient-derived CSF cell pellets, 13 were from patients with confirmed or possible RRMS, and 26 were from patients with OND. 14 patient samples (1 RRMS and 13 OND) were excluded due to recovery of insufficient sequence reads after sequence filtering resulting in a total of 26 samples available for analysis (Tables 5 and 6).

TABLE 4
Filtering by samples by cohort

| | Cohort name^{a} | | | |
|---|---|---|---|---|
| | RRMS | OND | HCN^{b} | TOTAL |
| Initial sample number | 13 | 26 | 10 | 40 |
| Samples with insufficient reads | 1^{c} | 13 | 0 | 14 |
| (<10 unique reads after filtering) | | | | |
| Total analyzed | 12 | 13 | 10 | 26 |

^{a}Samples were grouped into patient cohorts by final diagnosis.
^{b}Replicates from a single patient
^{c}Patient on Tysabri at time of sampling
Abbreviations:
RRMS, relapsing-remitting MS;
OND, other neurological disorder;
HCN, healthy control naive peripheral B cells.

We first determined how a series of process and analytical modifications we made since previous analyses affected sequence coverage. One modification was to include only unique sequences that had two or more copies after sequence filtering (redundancy 1) in an attempt to increase our confidence that the sequences being analyzed were representative of the B cell pool and not a result of sequence errors generated during either PCR amplification or NGS. Since the previous study did not include this filter, we compared the sequence coverage obtained with (R1) and without (RO) this redundancy filter (Table 7). In the previously published dataset we obtained an average of 2,426 unique sequences per RRMS sample at RO and an average of 583 sequences per RRMS sample at R1. In the current dataset an average of 751 sequences were obtained for the RRMS samples at R1 and an average of 632 sequences for the OND samples at R1 (Table 7). This resulted in a 1.3-fold increase per RRMS patient in the number of unique sequences in CSF-derived antibody repertoires using our current method over the previously published method (Rounds et al. Frontiers in Neurology 2014). The healthy control naive cohort had an average of 1,363 sequences per sample, which resulted in 2.5-fold more coverage in the peripheral healthy donor naive B cell pools in comparison to all CSF B cell pools, which likely relates to a larger initial pool of sorted B cells in the initial sample.

TABLE 5
RRMS sample summary

| | | | | | | Time | | |
|---|---|---|---|---|---|---|---|---|
| Patient | | | Diagnosis | | AGS | with | | OCB |
| ID | Age^{a} | Gender | at tap | Diagnosis^{b} | Score | MS^{c} | Treatment | Status^{a} |
| MS01 | 35 | F | RRMS | RRMS | 11.2 | NR | | NR |
| MS02 | 27 | F | RRMS | RRMS | −3.54 | 1 | Steroids (1 | POS |
| | | | | | | | month) | |
| MS03 | 39 | F | RRMS | RRMS | 33.25 | 0 | None | NEG |
| MS04 | 26 | F | RRMS | RRMS | 11.14 | 76 | Capaxone | NR |
| | | | | | | | (5 years) | |
| MS05 | 31 | F | RRMS | RRMS | 6.56 | 25 | Avonex (9 | POS |
| | | | | | | | months) | |
| MS06 | 42 | F | RRMS | RRMS | 10.02 | 92 | CellCept (7 | NEG |
| | | | | | | | years) | |
| MS07 | 35 | M | RRMS | RRMS | 26.82 | 24 | Avonex (2 | NEG |
| | | | | | | | years) | |
| MS08 | 36 | F | Possible | RRMS | 5.37 | 0 | | NR |
| | | | MS | | | | | |
| MS09 | 31 | M | RRMS | RRMS | 18.5 | 0 | None | NR |
| MS10 | 23 | F | Possible | RRMS | −1.51 | 0 | | NR |
| | | | MS | | | | | |
| MS11 | 32 | F | RRMS | RRMS | 15.22 | 0 | None | POS |
| MS12 | 58 | F | Possible | RRMS | 7.46 | 0 | ?? | NR?? |
| | | | MS | | | | | |

^{a}At time of sampling (yrs)
^{b}Most up-to-date available
^{c}At time of sampling (months)
^{d}If immunomodulatory and at time of sampling
*low unique sequence read count sample (<10)
Abbreviations:
NR, not reported;
OCB, oligoclonal bands;
RRMS, relapsing-remitting MS

TABLE 6
Non-RRMS sample summary.

| Patient ID | Age^{a} | Gender | Diagnosis^{b} |
|---|---|---|---|
| OND01 | 37 | M | OND |
| OND02 | 61 | M | Dementia |
| OND03 | 54 | M | Stroke |
| OND04 | 65 | F | Dementia |
| OND05 | 52 | F | Headache |
| OND06 | 48 | F | Neurosarcoidosis |
| OND07 | NR | F | Headache |
| OND08 | 57 | F | PND |
| OND09 | 44 | F | Encephalitis |
| OND10 | 67 | F | PND |
| OND11 | 49 | F | Urge incontinence |
| OND12 | 52 | M | Alzheimer's |
| OND13 | 22 | F | Headache |
| HCN | NR | NR | NA |

^{a}At time of sampling (yrs)
^{b}Most up-to-date available
*low unique sequence read count sample (<10)
Abbreviations:
NR, not reported;
OND, other neurological disorder;
PND, paraneoplastic neurologic disorder;
HCN, healthy control naive peripheral B cells.

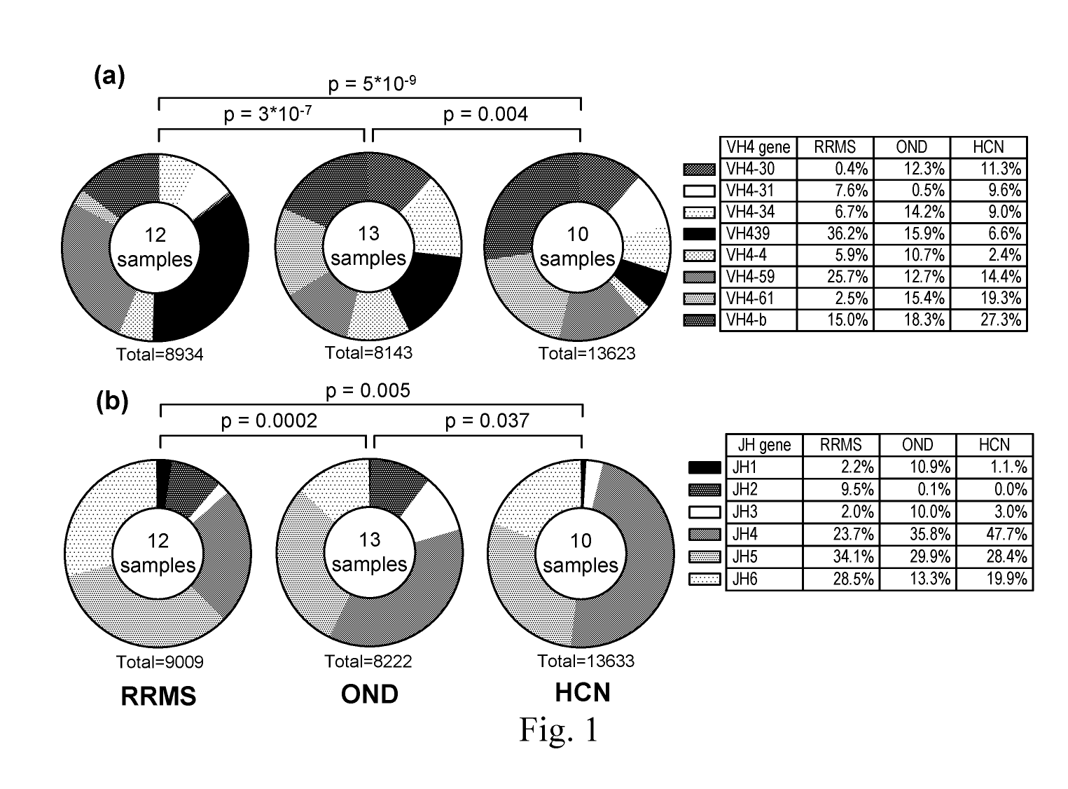

Next, we sought to determine if the distributions of variable heavy chain family 4 (VH4) gene segments in each cohort were comparable (FIG. 1a). The overall VH4 gene distribution of the RRMS cohort was significantly different from both the OND (p=3*10⁻⁷) and the healthy control naive (HCN) (p=5*10⁻⁹) cohorts. In contrast, the OND and HCN cohorts were more similar in distribution (p-value of 0.004). As expected, the usage frequency of VH4 genes in the HCN B cell pool was comparable to a random frequency of 12.5% for each individual gene. VH4-4 was significantly under-represented (p=0.007) and VH4-b was significantly over-represented (p=0.009) in the HCN cohort. Similarly, the OND cohort did not display a significant bias in VH4 gene usage as the majority of the VH4 gene frequencies were close to the random frequency of 12.5%, with the exception of VH4-31, which was under-represented (OND, 0.5%; RRMS, 7.6%; HCN, 9.6%, p=0.0006). In contrast, the RRMS cohort utilized VH4-39 (RRMS, 36.2% vs Random, 12.5%, p=9*10⁻⁵) and VH4-59 (RRMS, 25.7% vs Random 12.5%, p=0.02) more frequently than expected, which others have previously observed for VH4-39.

TABLE 7
Sequence yield per cohort

| | | Redundancy 0^{a} | | Redundancy 1^{b} | | | |
|---|---|---|---|---|---|---|---|
| | | | Avg. | | Avg. | | |
| | | | unique | | unique | | |
| | | Total | sequences | Total | sequences | Avg. RM | Avg. RM |
| Cohort | | unique | per | unique | per | per | per |
| name | N | sequences^{c} | sample | sequences | sample | sample | sequences |
| RRMS | 12 | 28,489 | 2,374 | 9,009 | 751 | 5,229 | 7.0 |
| OND | 13 | 20,201 | 1,554 | 8,222 | 632 | 3,025 | 4.8 |
| HCN | 10^{d} | 93,204 | 9,320 | 13,633 | 1,363 | 1,738 | 1.3 |
| Previous | 7 | 16,984 | 2,426 | 4,082 | 583 | 5,466 | 9.4 |
| MS | | | | | | | |

^{a}Sequence redundancy indicates additional copy number of a unique read (0 = sequence had no duplicates). At redundancy 0, no sequences are filtered out. SHown here for comparison with previous study indiated (Rounds et al. Frontiers in Neurology2014)
^{b}Filter used for this study. Each unique sequence read needs at least one matching read to pass.
^{c}After sequence filtering
^{d}Replicates from a single patient
Abbreviations:
RM, replacement mutation;
RRMS, relapsing-remitting MS;
OND, other neurological disorder;
HCN, healthy control naive peripheral B cells.

The distribution of junctional heavy chain (JH) gene segments in naïve B cells is heavily skewed towards JH4 usage, followed in rank by JH5 and JH6. The other JH segments occur at a lower frequency. Indeed, the healthy donor peripheral naïve B cell pools in the current dataset demonstrated skewing towards JH4 usage (FIG. 1b). However, the RRMS cohort for this dataset had a JH usage rank of 5>6>4>2>1=3. The high usage of JH5 and JH6 gene segments was unexpected and contrasted with the previous dataset where JH4 was maintained as the most frequently used JH gene segment in the RRMS cohort. Thus, the overall distribution of JH gene segments in the RRMS cohort was significantly different from that of the HCN cohort (p<0.005). The OND cohort for this dataset had a JH gene segment usage rank of 4>5>6>1=3>2, which more closely followed the JH rank of a naïve B cell repertoire (p=0.04).

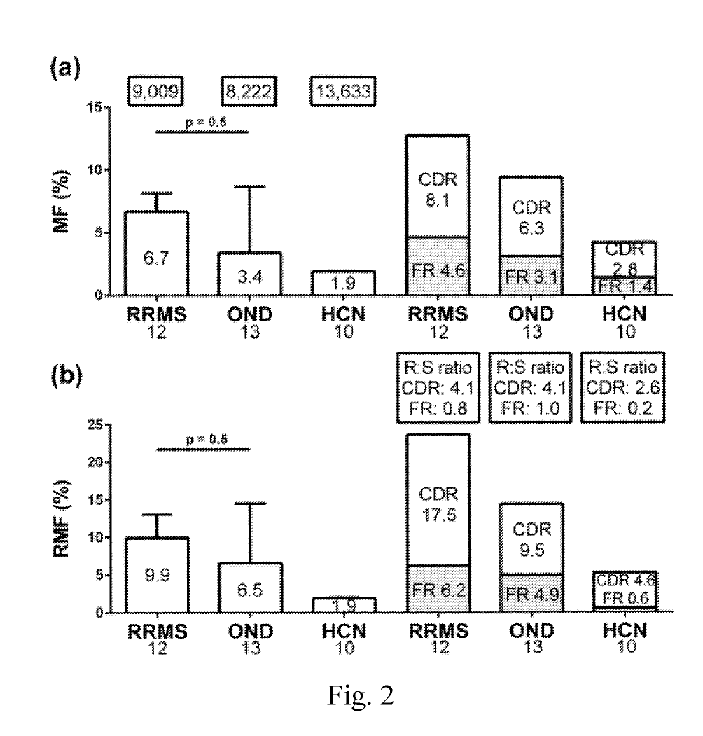

We next sought to determine whether the RRMS and OND cohorts from this dataset had accumulated somatic hypermutations (SHM) into the variable regions of their antibody genes as established in the literature. We did this by calculating both the overall mutation frequency (MF), which considers all nucleotide substitutions, and the replacement mutation frequency (RMF), which considers only those nucleotide substitutions that result in an amino acid replacement at each codon position (FIG. 2a). Whereas the healthy donor naïve B cell pools had very low MFs (median 1.9%), the RRMS and OND cohorts had very high MFs (medians 6.7% for RRMS and 3.4% for OND), demonstrating that CSF B cells accumulate SHM at a high frequency as has been previously published (Monson et al. J Neuroimmunol 2005). Interestingly, the MF of the RRMS and OND cohorts were not significantly different (p=0.50), even though the underlying pathology of the diseases for the patients in these two cohorts is expected to be quite distinct. The RMF calculations demonstrate a similar result (e.g., high and comparable RMF in the RRMS and OND CSF cohorts compared to the peripheral naïve). No correlation was found between patient age and RMF for either cohort (RRMS p=0.8; OND p=0.2). Proper targeting of these mutations to the hypervariable regions within the CDRs was confirmed since the majority of mutations overall and replacement mutations specifically occurred within the CDRs rather than the FRs (FIG. 2b).

Next, we compared the RMF at each codon position in the 6 codons that we originally used to calculate AGS scores (31B, 40, 56, 57, 81, 89). The RMF at codons 31B, 40, 56, and 57 were all statistically greater in the RRMS cohort compared to the OND cohort (Table 8). However, the RMF at codons 81 and 89 were statistically greater in the OND cohort compared to the RRMS cohort. In fact, codon 89 had the lowest RMF of all 6 AGS codons in the RRMS cohort (9.3%), and thus contributed the least to AGS scores for the RRMS cohort as a group as well as for individual patients.

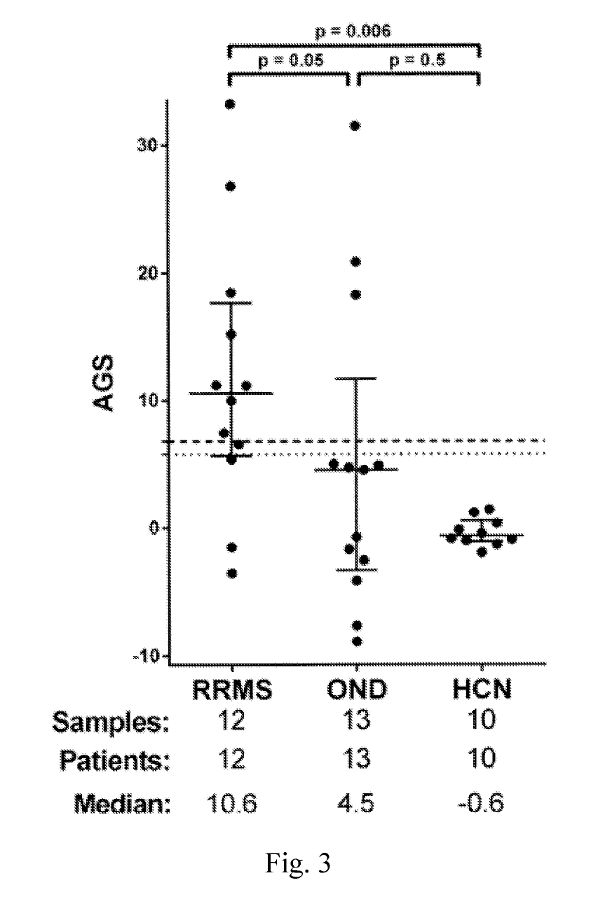

Finally, we calculated AGS scores for all 25 patient CSF samples and the 10 replicates of the HCN sample in the current dataset, excluding codon 89 in the calculations due to its low impact on AGS cores for the RRMS cohort (FIG. 3). The HCN cohort had very consistent AGS scores, with a median score of −0.6 and an interquartile range (IQR) of −1.1 to 0.6. As expected, the RRMS samples had statistically higher (p=0.006) AGS scores compared to HCN with a median score of 10.6 and IQR of 5.7 to 17.7. The OND samples tended to have intermediate AGS scores with a median score of 4.5 and IQR of −3.3 to 11.7, which is statistically less than the median AGS score of the RRMS cohort (p=0.05).

TABLE 8
AGS codon replacement mutation frequency relative
to germline in RRMS and OND patients

| AGS | | | OND | Fold higher in | |
|---|---|---|---|---|---|
| codon | Location | RRMS RMF^{a} | RMF | RRMS | p-value^{b} |
| 31B | CDR1 | 53.8% | 38.5% | 1.40 | 0.001 |
| 40 | FR2 | 16.4% | 13.0% | 1.26 | 0.001 |
| 56 | CDR2 | 33.6% | 15.7% | 2.14 | 0.001 |
| 57 | CDR2 | 21.1% | 4.8% | 4.42 | 0.001 |
| 81 | FR3 | 20.1% | 27.0% | 0.75 | 0.001 |
| 89 | FR3 | 9.3% | 13.7% | 0.68 | 0.001 |

^{a}Calculated relative to the total possible replacement mutations for each cohort (i.e., the number of reads that have a specific numbered codon in the germline)
^{b}Calculated by Chi-squared test
Abbreviations:
RMF, replacement mutation frequency;
RRMS, relapsing-remitting MS;
OND, other neurological disorder.

As expected, ten of 13 OND patients had AGS scores below the previously established threshold of 6.8. However, since the 6.8 threshold was based on Sanger sequencing data and NGS sequences have a low level of background RMs which has a tendency to lower AGS scores, we identified an alternative threshold of 5.8 where we would expect to find some NGS samples with AGS scores above but close to the threshold by Sanger sequencing. This new threshold did not affect the number of OND patients that had AGS scores low enough to be properly identified. Four of them had AGS scores just below the indeterminate AGS threshold, and 6 of them had negative AGS scores. The four OND patients with mid-level AGS scores were diagnosed with headache (n=1; AGS=4.91), neurosarcoidosis (n=1; AGS=4.74), Alzheimer's disease (n=1; AGS=5.04), and unspecified OND (n=1; AGS=4.55). The 6 OND patients with negative AGS scores were diagnosed with headache (n=2; AGS=−4.12/−8.89), paraneoplastic disease (n=2; AGS=−7.66/−2.55), dementia (n=1; AGS=−0.71) and encephalitis (n=1; AGS=−1.67). The 3 patients with scores above 5.8 were diagnosed with dementia (OND04; AGS=31.6), stroke (OND03; AGS=18.3), and urge incontinence (OND11; AGS=20.9).

AGS scores for 9 of the 12 RRMS patients were above the new AGS threshold of 5.8 and included 2 patients who were on Avonex (one for 9 months,MS05, and one for 2 years,MS07), one patient who was on Copaxone for 5 years (MSO4) and one patient who was on Cellcept for 7 years (MS06). All three of the patients diagnosed with RRMS who were OCB negative had AGS scores above the threshold (AGS scores=33.2; 10.0; 26.8), two of which were on DMT (MS06, Cellcept; MS07, Avonex). Of the three RRMS patients who were OCB positive, two had AGS scores below the threshold (AGS scores=−3.5 and 6.6), but had been sampled while on DMTs (MS02, steroids; MS05, Avonex). One OCB positive RRMS patient who was not on DMT at the time of sampling had an AGS score above the threshold (AGS score=15.2).

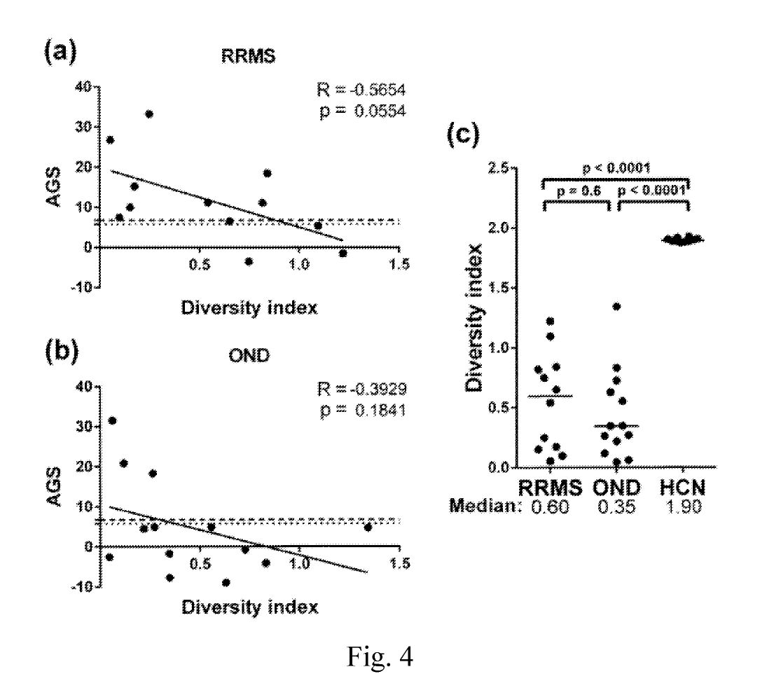

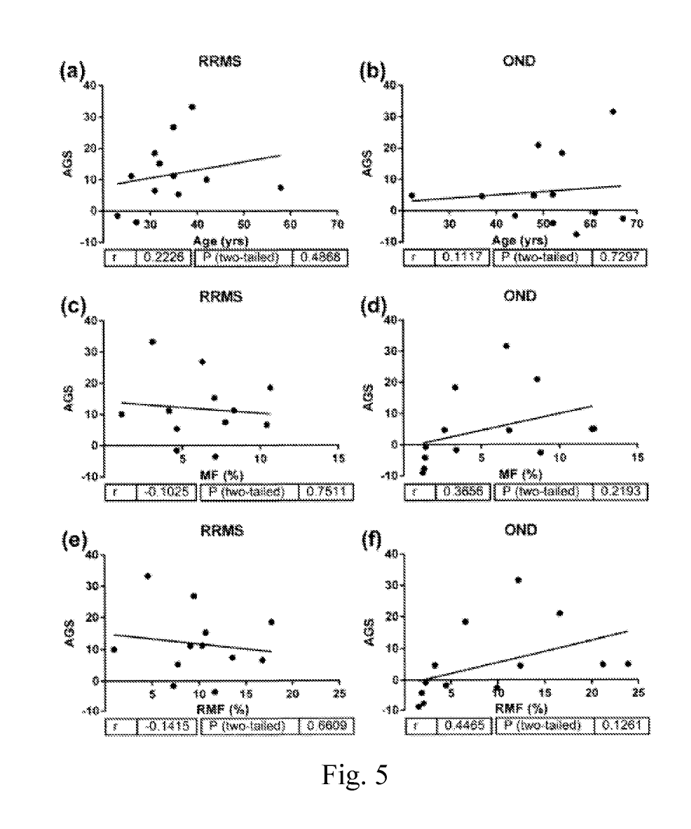

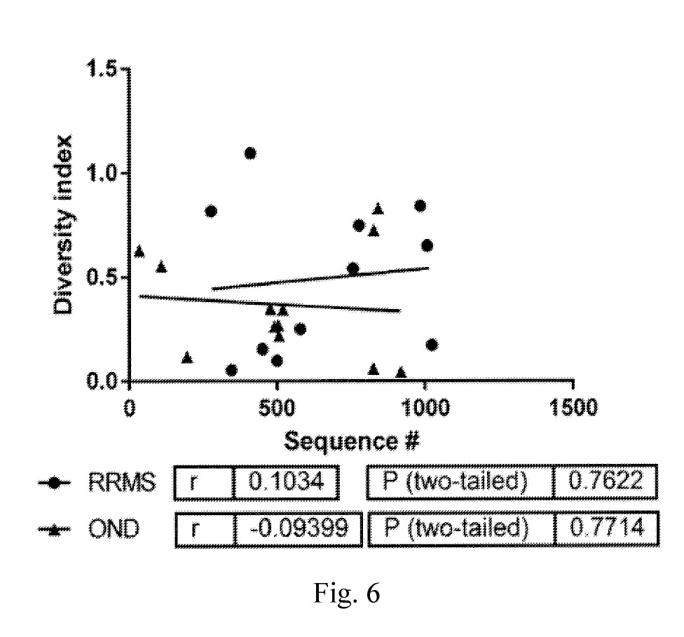

No correlations were found between AGS score and age or mutation frequency for either the RRMS or OND cohorts (FIG. 5). However, we did note a trend towards higher diversity in VH4 gene usage (termed “diversity index”) for RRMS patients with low AGS scores (FIG. 4a), which did not correlate with sequence read count (FIG. 6). The two RRMS patients that had high diversity indices and low AGS scores were MS08 (diversity index=1.10; AGS score=5.37) and MS10 (diversity index=1.22; AGS score=−1.51). The OND cohort did not display any correlation of AGS score with the diversity index (FIG. 4b), even though the diversity index for the RRMS and OND cohorts were not statistically different (FIG. 4c; p=0.6). The HCN cohort displayed a high diversity index that was statistically different from both the RRMS and OND cohorts (p<0.0001 for both) (FIG. 4c).

Discussion

The application of antibody genetics to human disease has begun to emerge rapidly, particularly since next generation sequencing (NGS) became readily available. Indeed, the power of this technology has been applied to monitoring minimal residual disease in cases of B cell lymphomas, and establishing that CSF-derived B cell clones do indeed matriculate from the peripheral blood. Our application of NGS has been in the arena of MS biomarkers, using it as a new approach to identify patients with clinically isolated syndromes (CIS) who are at high risk of converting to fulminant MS in the future. Indeed, our early work using Sanger DNA sequencing methods demonstrated that Antibody Gene Signature (AGS) scoring, which enumerates the prevalence of replacement mutations at specific codon positions in the rearranged variable gene segments of antibody genes, identified CIS patients who later converted to definite RRMS with 91% accuracy

However, four questions remained. First, was the accumulation of somatic hypermutations (SHM) in these codons specific to MS patients? Second, would RRMS patients that meet the revised McDonald criteria and who have had a confirmed diagnosis for an extended period of time, e.g. years, have a similar pattern of SHM as early-stage patients? Three, does OCB status affect the score? Four, does treatment with immunomodulating drugs affect the score? To address these issues, we generated antibody gene repertoires from CSF-derived B cells of ONDs, OCB and OCW RRMS patients as well as treatment-naïve RRMS patients and RRMS patients who had been on DMTs for more than a year.

We obtained CSF cell pellets from 26 OND patients with a variety of diagnoses including headache (n=6), paraneoplastic disease (n=4), dementia (n=3), stroke (n=1), neurosarcoidosis (n=1), encephalitis (n=1), Alzheimer's (n=1) urge incontinence (n=1), amyothrophic lateral sclerosis (n=1), chronic inflammatory demyelinating polyneurophathy (n=1), glioma (n=1), peripheral neuropathy (n=1), sensory neuropathy (n=1), Hodgkin's lymphoma (n=1) and nondemyelinating optic neuropathy (n=1). Of the 26 OND patients, 13 were excluded from analysis due to recovery of a very low number of sequence reads. Since this primarily occurred in the OND cohort, we concluded that those 13 OND patients either did not display an expanded B cell mediated CNS immune response that we could detect, or that the response was negligible. In either case, we believe that the inability to recover antibody sequences from such samples is indicative of a lack of B cell recruitment in those cases, and confirms why the literature is limited in the area of antibody genetics in patients with non-inflammatory neurological diseases. In fact, there was one RRMS patient with insufficient reads that we did not re-insert into the cohort because this patient had been on Tysabri for more than 4 years and thus would not have been in this analysis initially as Tysabri is well-known to prevent the extravasation of B cells from the periphery into the CNS.

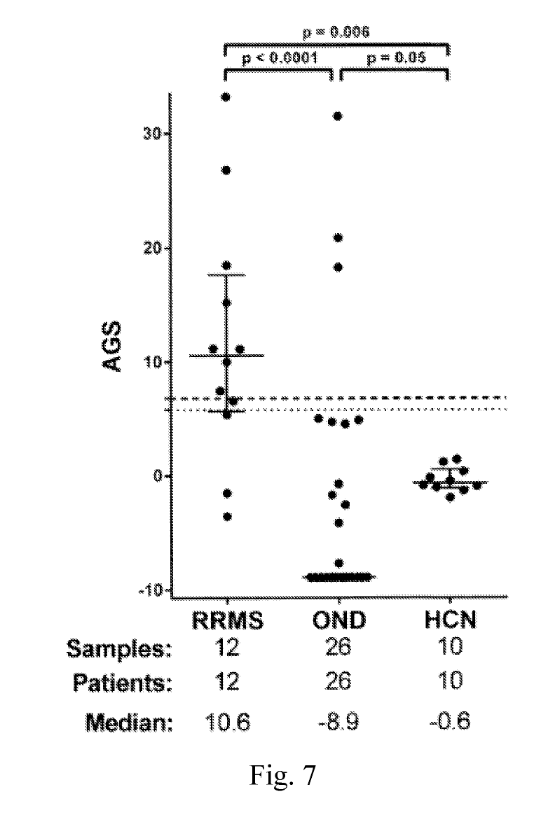

We were concerned that our ability to detect antibody genes of rare B cells by PCR might provide OND samples an advantage and the AGS scoring approach might not properly reflect their OND status. Removing samples from the analysis based on their low antibody sequence reads might actually be indicative of their OND status. Indeed, of the 14 samples we removed based on recovery of an insufficient number of unique sequence reads, 13 of them were within the OND cohort. If we assigned such samples the lowest AGS score possible (AGS score=−8.9), and inserted them back into the OND cohort, the median AGS score of the OND group decreases to −8.9 (FIG. 7).

In those 13 OND cases where we were able to recover a sufficient number of unique antibody sequences from CSF-derived cells, we observed that the accumulation of replacement mutations into their B cell pools was slightly lower than in the RRMS patients. However, the cohorts were not significantly different (OND, median RMF 6.5; RRMS, median RMF 9.9; p=0.5). In addition, the distribution of VH4 gene segments in the OND cohort was fairly equal such that 7 of the 8 VH4 genes displayed frequencies that did not differ significantly from the expected random frequency. JH gene segment usage was also no different from the expected frequency established in naïve B cell pools. This suggests that in the OND cases for which CSF B cells can be detected, antigen-driven selection is not as apparent as it is in the CSF collected from RRMS patients.

There is very little available information regarding the impact of DMTs on numbers or types of B cells found in the CSF of RRMS patients. Even in the case of B cell-depleting monoclonal antibodies, such as Rituximab, our understanding of B cell dynamics in the CSF is limited. Nevertheless, the RRMS cohort used for this study included 4 patients on DMTs, most of which had high AGS scores regardless of OCB status. The one RRMS patient on steroids at the time of sampling had a negative AGS score. It is difficult to make conclusions based on these small samples, but this data suggests that the clinical benefit of many immunomodulatory drugs used to treat RRMS, including the beta interferons and Copaxone, is independent of the CSF B cell pool.

Finally, there is an increasing need for new methods to determine whether a patient has MS or not. AGS scoring may be one supportive approach to aid clinicians in this task. Indeed, if we include the OND samples with insufficient reads, the specificity of identifying patients with OND based on AGS scoring is 88%. The sensitivity of this test in identifying RRMS patients is 75%, although the impact of DMTs and steroids on the AGS scoring system for our RRMS cohort remains unclear. This puts the overall accuracy of AGS scoring in this study at 84% if samples with insufficient reads are included and 76% if they are omitted. Previously, we presented data generated using Sanger DNA sequencing suggesting that AGS scoring is able to identify CIS patients who will convert to RRMS but who are not yet on immunomodulatory therapy with 91% accuracy.

### REFERENCES, EACH OF WHICH IS INCORPORATED BY REFERENCE IN ITS ENTIRETY

1. Andersson M, Alvarez-Cermeno J, Bernardi G, et al. Cerebrospinal fluid in the diagnosis of multiple sclerosis: a consensus report. Journal of neurology, neurosurgery, and psychiatry. 1994; 57: 897-902.

2. Owens G P, Bennett J L, Gilden D H and Burgoon M P. The B cell response in multiple sclerosis. Neurological research. 2006; 28: 236-44.

3. Harp C, Lee J, Lambracht-Washington D, et al. Cerebrospinal fluid B cells from multiple sclerosis patients are subject to normal germinal center selection. Journal of neuroimmunology. 2007; 183: 189-99.

4. Owens G P, Winges K M, Ritchie A M, et al. VH4 gene segments dominate the intrathecal humoral immune response in multiple sclerosis. J Immunol. 2007; 179: 6343-51.

5. Bennett J L, Haubold K, Ritchie A M, et al. CSF IgG heavy-chain bias in patients at the time of a clinically isolated syndrome. Journal of neuroimmunology. 2008; 199: 126-32.

6. Antel J and Bar-Or A. Roles of immunoglobulins and B cells in multiple sclerosis: From pathogenesis to treatment. Journal of neuroimmunology. 2006; 180: 3-8.

7. Lambracht-Washington D, O'Connor K C, Cameron E M, et al. Antigen specificity of clonally expanded and receptor edited cerebrospinal fluid B cells from patients with relapsing remitting MS. Journal of neuroimmunology. 2007; 186: 164-76.

8. Hauser S L, Waubant E, Arnold D L, et al. B-cell depletion with rituximab in relapsing-remitting multiple sclerosis. N Engl J Med. 2008; 358: 676-88.

9. Kappos L, Li D, Calabresi P A, et al. Ocrelizumab in relapsing-remitting multiple sclerosis: a phase 2, randomised, placebo-controlled, multicentre trial. Lancet. 2011; 378: 1779-87.

10. Qin Y, Duquette P, Zhang Y, Talbot P, Poole R and Antel J. Clonal expansion and somatic hypermutation of V(H) genes of B cells from cerebrospinal fluid in multiple sclerosis. J Clin Invest. 1998; 102: 1045-50.

11. von Budingen H C, Harrer M D, Kuenzle S, Meier M and Goebels N. Clonally expanded plasma cells in the cerebrospinal fluid of MS patients produce myelin-specific antibodies. Eur J Immunol. 2008; 38: 2014-23.

12. von Budingen H C, Kuo T C, Sirota M, et al. B cell exchange across the blood-brain barrier in multiple sclerosis. J Clin Invest. 2012; 122: 4533-43.

13. Cameron E M, Spencer S, Lazarini J, et al. Potential of a unique antibody gene signature to predict conversion to clinically definite multiple sclerosis. J Neuroimmunol. 2009; 213: 123-30.

14. Rounds W H, Ligocki A J, Levin M K, et al. The antibody genetics of multiple sclerosis:

comparing next-generation sequencing to sanger sequencing. Frontiers in neurology. 2014; 5: 166.

15. Polman C H, Reingold S C, Banwell B, et al. Diagnostic criteria for multiple sclerosis: 2010 revisions to the McDonald criteria. Ann Neurol. 2011; 69: 292-302.

16. Chothia C and Lesk A M. Canonical structures for the hypervariable regions of immunoglobulins. Journal of molecular biology. 1987; 196: 901-17.

17. Al-Lazikani B, Lesk AM and Chothia C. Standard conformations for the canonical structures of immunoglobulins. Journal of molecular biology. 1997; 273: 927-48.

18. Kabat E A, Te Wu T, Perry H M, Gottesman K S and Foeller C. Sequences of proteins of immunological interest. DIANE publishing, 1992.

19. Brezinschek H P, Brezinschek R I and Lipsky P E. Analysis of the heavy chain repertoire of human peripheral B cells using single-cell polymerase chain reaction. J Immunol. 1995; 155: 190-202.

20. Owens G P, Kraus H, Burgoon M P, Smith-Jensen T, Devlin M E and Gilden D H. Restricted use of VH4 germline segments in an acute multiple sclerosis brain. Ann Neurol. 1998; 43: 236-43.

21. Baranzini S E, Jeong M C, Butunoi C, Murray R S, Bernard C C and Oksenberg J R. B cell repertoire diversity and clonal expansion in multiple sclerosis brain lesions. J Immunol. 1999; 163: 5133-44.

22. Monson N L, Brezinschek H P, Brezinschek R I, et al. Receptor revision and atypical mutational characteristics in clonally expanded B cells from the cerebrospinal fluid of recently diagnosed multiple sclerosis patients. Journal of neuroimmunology. 2005; 158: 170-81.

23. Boyd S D, Marshall E L, Merker J D, et al. Measurement and clinical monitoring of human lymphocyte clonality by massively parallel VDJ pyrosequencing. Sci Transl Med. 2009; 1: 12ra23.

24. Stuve O and Bennett J L. Pharmacological properties, toxicology and scientific rationale for the use of natalizumab (Tysabri) in inflammatory diseases. CNS Drug Rev. 2007; 13: 79-95.

25. Evdoshenko E, Maslyanskiy A, Lapin S, et al. Dynamics of B-Cell Populations in CSF and Blood in Patients Treated with a Combination of Rituximab and Mitoxantrone. ISRN neurology. 2013; 2013: 748127.

26. Kroksveen A C, Opsahl J A, Guldbrandsen A, et al. Cerebrospinal fluid proteomics in multiple sclerosis. Biochimica et biophysica acta. 2014.ens GP, Bennett J L, Gilden D H and Burgoon M P. The B cell response in multiple sclerosis. Neurological research. 2006; 28: 236-44.

27. Ireland S J, Guzman A A, O'Brien D E, et al. The effect of glatiramer acetate therapy on functional properties of B cells from patients with relapsing-remitting multiple sclerosis. JAMA neurology. 2014; 71: 1421-8.

28. Ye J, Ma N, Madden T L and Ostell J M. IgBLAST: an immunoglobulin variable domain sequence analysis tool. Nucleic acids research. 2013; 41: W34-40.

29. Jackson K J, Kidd M J, Wang Y and Collins A M. The Shape of the Lymphocyte Receptor Repertoire: Lessons from the B Cell Receptor. Front Immunol. 2013; 4: 263.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A method comprising:
(a) amplifying a region comprising two or more codons of a set of variable heavy (VH)4 antibody genes from a nucleic acid sample produced from a subject sample;
(b) sequencing the amplified regions using next generation sequencing to generate a set of sequence reads;
(c) processing the set of sequence reads to generate a set of (VH)4 sequences; and
(d) selecting the subject sample as suitable for diagnostic testing, reporting, or diagnostic testing and reporting when one or more of the following sample quality indicators are met:
(i) the set of (VH)4 sequences are from more than a first threshold number of (VH)4 genes,
(ii) the set of (VH)4 sequences are from a second threshold number to the first threshold number of (VH)4 antibody genes, and a diversity index for the set of (VH)4 sequences is greater than a diversity index threshold, wherein the second threshold number is less than the first threshold number,
(iii) greater than or equal to a first threshold percentage of the set of sequence reads are (VH)4 sequences,
(iv) less than or equal to a second threshold percentage of the set of sequence reads contain a CDR3 sequence identical to another sample, or
(v) a composite signature score for the set of (VH)4 sequences is not an indeterminate result.

2. The method of claim 1, wherein the subject sample is selected when the set of (VH)4 sequences are from more than the first threshold number of (VH)4 genes.

3. The method of claim 1, wherein the subject sample is selected when the set of (VH)4 gene sequences are from the second threshold number to the first threshold number of (VH)4 genes, and the diversity index for the set of (VH)4 sequences is greater than the diversity index threshold.

4. The method of any one of claims 1-3, wherein the subject sample is selected when greater than or equal to the first threshold percentage of the set of sequence reads are (VH)4 sequences.

5. The method of any one of claims 1-4, wherein the subject sample is selected when less than or equal to the second threshold percentage of the set of sequence reads contain a CDR3 sequence identical to another sample.

6. The method of any one of claims 1-5, wherein the subject sample is selected when the composite signature score for the set of (VH)4 sequences is not an indeterminate result.

7. The method of any one of claims 1-6, wherein the subject sample is selected when two or more of the sample quality indicators are met.

8. The method of any one of claims 1-6, wherein the subject sample is selected when three or more of the sample quality indicators are met.

9. The method of any one of claims 1-6, wherein the subject sample is selected when four of the sample quality indicators are met.

10. The method of any one of claims 1-9, wherein the first threshold number of (VH)4 genes is about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

11. The method of any one of claims 1-9, wherein the first threshold number of (VH)4 genes is about 30.

12. The method of any one of claims 1-9, wherein the first threshold number of (VH)4 genes is about: 10-50, 20-40, or 25-35.

13. The method of any one of claims 1-9, wherein the first threshold number of (VH)4 genes is about: 25-35.

14. The method of any one of claims 1-13, wherein the second threshold number of (VH)4 genes is about: 1, 2, 3, 4, 5, 6, 7, 8, or 9.

15. The method of any one of claims 1-13, wherein the second threshold number of (VH)4 genes is about 5.

16. The method of any one of claims 1-13, wherein the second threshold number of (VH)4 genes is about: 1-9, 2-8, 3-7, or 4-6.

17. The method of any one of claims 1-13, wherein the second threshold number of (VH)4 genes is about 4-6.

18. The method of any one of claims 1-17, wherein the diversity index (H′) is calculated using the following formula:
$${H}^{′} = - {\sum }_{i = 1}^{R} {p}_{i} ln {p}_{i}$$
wherein pᵢ is the proportion of the total number of VH4 sequences within a given VH4 antibody subfamily, and R is the total number of species in the subfamily.

19. The method of claim 18, wherein the diversity index threshold is about: 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

20. The method of claim 18, wherein the diversity index threshold is about 1.0.

21. The method of claim 18, wherein the diversity index threshold is about: 1.0-5.0, 1.0-4.0, 1.0-3.0, 1.0-2.5, 1.0-2.0, or 1.0-1.5.

22. The method of claim 18, wherein the diversity index threshold is about 0.85-1.15.

23. The method of any one of claims 1-22, wherein the first threshold percentage is about: 5%, 10%, 15%, 20%, 25%, 30%, 35%, 40%, 45%, 50%, 55%, 60%, 65%, 70%, 75%, 80%, 85%, 90%, 95% or 99%.

24. The method of any one of claims 1-22, wherein the first threshold percentage is about 55%.

25. The method of any one of claims 1-22, wherein the first threshold percentage is about 60%.

26. The method of any one of claims 1-22, wherein the first threshold percentage is about: 5-99%, 10-95%, 15-90%, 20-85%, 25-80%, 30-75%, 35-70%, 40-65%, 45-60%, or 50-60%.

27. The method of any one of claims 1-22, wherein the first threshold percentage is about 40-65%.

28. The method of any one of claims 1-22, wherein the first threshold percentage is about 50-60%.

29. The method of any one of claims 1-28, wherein the second threshold percentage is about: 5%, 10%, 15%, 20%, 25%, 30%, 35%, 40%, 45%, 50%, 55%, 60%, 65%, 70%, 75%, 80%, 85%, 90%, 95% or 99%.

30. The method of any one of claims 1-28, wherein the second threshold percentage is about 50%.

31. The method of any one of claims 1-28, wherein the second threshold percentage is about: 5-99%, 5-95%, 10-90%, 15-85%, 20-80%, 25-75%, 30-70%, 40-60%, or 45-55%.

32. The method of any one of claims 1-28, wherein the second threshold percentage is about 40-60%.

33. The method of any one of claims 1-28, wherein the second threshold percentage is about 45-55%.

34. The method of any one of claims 1-33, wherein the composite signature score is the sum of replacement mutation frequencies at two or more codon positions.

35. The method of claim 34, wherein the replacement mutation frequencies at individual codon positions are normalized by subtracting the average replacement mutation frequency at the same codon position from a healthy control population and dividing by the standard deviation of the average mutation frequency for the same codon position in the healthy control population.

36. The method of claim 34 or 35, wherein the two or more codon positions are selected from the group consisting of 31B, 40, 56, 57, 81, and 89.

37. The method of claim 34 or 35, wherein the two or more codon positions are selected from the group consisting of 31B, 40, 56, 57, and 81.

38. The method of claim 34 or 35, wherein the two or more codon positions are 31B, 40, 56, 57, and 81.

39. The method of any one of claims 1-38, wherein the indeterminate result is a composite signature score of about 0.8-10.8, 1.8-9.8, 2.8-8.8, 3.8-7.8, 4.8-6.8, 0.8-12.8, 1.8-11.8, 2.8-10.8, 3.8-9.8, 4.8-8.8, or 5.8-7.8.

40. The method of any one of claims 1-38, wherein the indeterminate result is a composite signature score of about 4.8-6.8.

41. The method of any one of claims 1-38, wherein the indeterminate result is a composite signature score of about 5.8-7.8.

42. The method of any one of claims 1-41, wherein the sample comprises beta cells.

43. The method of any one of claims 1-42, wherein the sample comprises cerebral spinal fluid, blood, or a combination thereof.

44. The method of any one of claims 1-42, wherein the sample comprises cerebral spinal fluid.

45. The method of any one of claims 1-42, wherein the sample comprises a cell pellet from cerebral spinal fluid.

46. The method of any one of claims 1-43, wherein the sample is from a subject suspected of having, or being at risk of developing, a neurological disorder.

47. The method of any one of claims 1-46, wherein the sample is from a subject suspected of having, or being at risk of developing, multiple sclerosis.

48. The method of claim 47, wherein the multiple sclerosis is relapsing-remitting multiple sclerosis.

49. The method of any one of claims 1-48, wherein the nucleic acid sample comprises DNA, RNA or a combination thereof.

50. The method of any one of claims 1-48, wherein the nucleic acid sample comprises genomic DNA, mRNA, cDNA, or a combination thereof.

51. The method of any one of claims 1-48, wherein the nucleic acid sample comprises genomic DNA.

52. The method of any one of claims 1-48, wherein the nucleic acid sample comprises whole genome amplified DNA.

53. The method of any one of claims 1-52, wherein the amplifying comprises specifically hybridizing primers to the region.

54. The method of any one of claims 1-53, wherein the region comprises codons 24 to 95 of the set of variable heavy (VH)4 antibody genes.

55. The method of any one of claims 1-53, wherein the region comprises codons 31 to 91 of the set of variable heavy (VH)4 antibody genes.

56. The method of any one of claims 1-55, wherein next generation sequences comprises 454 sequencing, pyrosequencing, SOLid sequencing, SOLEXA sequencing, SMRT sequencing, nanopore sequencing, ion semiconductor sequencing, DNA nonoball sequencing, or tSMS sequencing.

57. The method of any one of claims 1-55, wherein next generation sequences comprises 454 sequencing.

58. The method of any one of claims 1-57, wherein processing the set of individual sequence reads comprises trimming sequences to remove primer sequences, trimming sequences to remove sample barcode sequences, aligning individual sequence reads to each other to identify unique sequences, aligning unique sequences to germline gene segment sequences, removing low quality sequences, removing sequences containing CDR3 sequences that align to a CDR3 sequence from another sample, or a combination thereof.

59. The method of any one of claims 1-57, wherein processing the set of individual sequence reads comprises aligning unique sequences to germline gene segment sequences.

60. The method of any one of claims 1-59, further comprising, if the sample is selected as suitable for diagnostic testing and reporting, providing a report to a party comprising one or more of: a composite signature score, a diagnosis of having or not having MS, the results of one or more other laboratory tests, or a combination thereof.

61. The method of claim 60, wherein the one or more other laboratory tests include an oligoclonal banding test, an MRI result or image, or a combination thereof.

62. The method of claim 60 or 61, wherein the report is provided via a communication medium.
